(19) United States
(12) Patent Application Publication
(10) Pub. No.: US 2024/0108508 A1
(43) Pub. Date: Apr. 4, 2024

(54) METHOD AND SYSTEM FOR FORMING INTRACORNEAL CUTS USING A CONVEX CONTACT SURFACE

(71) Applicant: TECHNOLAS PERFECT VISION GMBH, MUENCHEN (DE)

(72) Inventors: Wolfgang KÜGEL, Karlsfeld (DE); Christof DONITZKY, Eckental (DE)

(21) Appl. No.: 18/532,559

(22) Filed: Dec. 7, 2023

Related U.S. Application Data
(63) Continuation of application No. PCT/EP2022/065423, filed on Jun. 7, 2022.

(30) Foreign Application Priority Data
Jun. 7, 2021 (EP) 21178105.9
Jun. 7, 2022 (WO) PCT/EP2022/065423

(51) Int. Cl. A61F 9/008; A61F 9/009

(52) U.S. Cl. CPC A61F 9/00836; A61F 9/009; A61F 2009/00872

(57) ABSTRACT

The present disclosure relates to an eye treatment system for performing laser surgery on an eye which includes a laser optical system. The laser optical system includes a scanning system for scanning a focus of a laser beam of the laser light within a cornea of the eye in three dimensions. The laser optical system further includes a focusing optical system. The scanning system is in the beam path of the laser beam between the laser source and the focusing optical system. The eye treatment system further includes a contact element which is in the beam path of the laser beam between the focusing optical system and the eye. The contact element has a contact surface for contacting a cornea of the eye. At least a portion of the contact surface has a shape, which is convex toward the cornea.

### FIELD

The present invention relates to a system and a method for forming cuts within of a cornea of the eye. Specifically, the present invention relates to a system and a method for applying laser light to an exposed stromal surface for forming a hinged flap or an intrastromal lenticule.

### BACKGROUND

Laser-assisted in-situ keratomileusis (LASIK) is one of the most common laser surgeries. The surgery involves forming a hinged corneal flap. The hinge of the flap allows peeling back the flap to establish access to the stromal tissue that is to be ablated using an excimer laser beam which is focused directly onto the exposed corneal stroma in a pattern to correct the refractive-error.

In established flap formation procedures, the flap is formed using a mechanical microkeratome (which has an oscillating blade designed for cutting the hinged flap) or a focused femtosecond laser beam. The femtosecond laser has become more popular than microkeratome procedures because of its greater accuracy and predictability. Specifically, femtosecond laser allows creation of highly predictable, reproducible and stable corneal flaps within a narrow range of intended flap thickness and diameter. In comparison, conventional microkeratomes generally generate flaps that are thinner in the center compared to the periphery, which can lead to buttonhole perforations. Also, the femtosecond laser allows formation of cuts which have a higher degree of stromal bed smoothness.

However, femtosecond laser-assisted LASIK still has complications, which do not occur in microkeratome procedures. One of the effects, which may lead to complications is the occurrence of cavitation bubbles, which are caused by the photodisruption of corneal tissue in the focal is of the laser beam. The vaporized tissue forms cavitation gas bubbles, which collapse and leave behind gas bubbles, which consist of carbon dioxide (CO₂), nitrogen (N₂) and water (H₂O) as main constituents. Accumulation of the gas bubbles in the superficial layers of the stromal bed can lead to so-called opaque bubble layers (OBL), which create a diffuse opacity.

Excessive OBLs can lead to interference in many stages of the surgical procedure such as flap creation, flap lifting, residual stromal bed measurements and laser tracking for the excimer laser ablation process. A further complication, which is related to cavitation gas bubbles is the occurrence of vertical gas breakthroughs (VGB). VGBs may lead to incomplete separation cuts between the flap and the stromal bed to the extent that holes in the flap are generated when the surgeon tries to fold the flap back. It is also possible that the gas bubbles migrate into the anterior chamber of the eye, where they can interfere with the excimer laser eye trackers.

For LASIK surgical procedures, it is also desirable to have a laser process, which allows formation of a smooth stromal bed cut, that provides a well-defined stromal surface for ablation with the excimer laser and is sufficiently fast so that the surgical procedure is not stressful for the patient and the occurrence of errors caused by excessive movement of the patient is not increased.

Similar problems exist in processes for forming intrastromal lenticules which later will be separated from the cornea either through a small incision (small incision lenticule extraction, abbreviated as SMILE hereinafter) or using a hinged flap (femtosecond lenticule extraction, abbreviated as FLEx hereinafter). Also techniques, in which a laminar portion of the cornea is replaced by a transplant (such as in lamellar keratoplasty) have similar problems.

Accordingly, a need exists for an improved laser system and eye treatment methods, which overcome one or more of the above discussed problems.

### SUMMARY

Embodiments of the present disclosure pertain to an eye treatment system for performing laser surgery on an eye. The system comprises a laser optical system having a laser source configured to generate pulsed laser light having a pulse duration of less than 1 picosecond. The laser optical system comprises a scanning system for scanning a focus of a laser beam of the laser light within a cornea of the eye in three dimensions. The eye treatment system further comprises a contact element, which is in the beam path of the laser beam between the focusing optical system and the eye. The contact element has a contact surface for contacting a cornea of the eye wherein at least a portion of the contact surface has a shape, which is convex toward the cornea.

According to an embodiment, the eye treatment system comprises a focusing optical system, wherein the scanning system is in the beam path of the laser beam between the laser source and the focusing optical system.

The laser system may be configured for laser surgery on an eye, in particular for generating cuts within a cornea of the eye using a pulsed laser beam. The laser source may be configured so that the laser pulses have a pulse energy so that the laser beam generates photodisruption within corneal tissue. The photodisruption may be caused by laser-induced optical breakdown. Alternatively, a pulse energy of the laser pulses may be below a threshold for generating laser-induced optical breakdown. By way of example, a plurality of pulses, which have a pulse energy below the threshold for generating laser-induced optical breakdown may be overlapped in a manner so as to generate tissue separation within the cornea.

The laser source may be configured so that a pulse energy of the laser pulses is greater than 1 nanojoule, or greater than 10 nanojoule or greater than 50 nanojoule. The pulse energy may be less than 20 microjoule, or less than 15 microjoule or less than 10 microjoule.

A pulse duration of the pulsed laser beam may be less than 800 femtoseconds, or less than 500 femtoseconds, or less than 300 femtoseconds, or less than 150 femtoseconds, or less than 100 femtoseconds. The pulse duration may be greater than 10 femtoseconds or greater than 50 femtoseconds. A repetition rate of the pulsed laser beam may be greater than 50 kHz or greater than 80 kHz. The repetition rate of the pulsed laser beam may be less than 10 MHz or less than 1 MHz.

A center wavelength of the pulsed laser beam, which is incident on the eye may be in a range of between 800 nanometers and 1400 nanometers, or between, 900 nanometers and 1400 nanometers, or between 1000 nanometers and 1100 nanometers, or between 1010 nanometers and 1050 nanometers.

The laser source may include a pre-compensator for at least partially pre-compensating a change of the group delay dispersion (GDD) of the laser pulses, which is induced by components of the laser optical system, which are in the beam path of the laser beam downstream of the laser source. If a laser pulse has a positive GDD, longer wavelengths of the laser pulse propagate faster than shorter wavelengths. A positive group delay dispersion therefore corresponds to a material dispersion, which is typical in transparent media, since red wavelengths experience a lower refractive index compared to blue wavelengths. The pre-compensator may be configured to reduce the group delay dispersion. By way example, the reduced group delay dispersion generated by the pre-compensator may have a lower positive or a more negative group delay dispersion.

A lateral diameter of the focus of the laser beam within the cornea may be smaller than 10 micrometers, or smaller than 6 micrometers. The diameter may be greater than 3 micrometers. The lateral diameter may be measured in a direction perpendicular to an optical axis of the laser optical system. The lateral diameter may be measured as an 80% encircled energy diameter.

The laser optical system may include a controller for controlling the laser optical system. The controller may include a data processing system. The data processing system may include a computer system having a processor and a memory for storing instructions processable by the processor. The processor may execute an operating system. The data analysis system may further include a user interface configured to allow a user to receive data from the data processing system and/or to provide data to the data processing system. The user interface may include a graphical user interface.

The controller may be configured to determine a scanning path of the pulsed laser beam for scanning the laser focus within the cornea. The controller may be configured to determine the scanning path based on patient specific data. By way of example, the controller may be configured to determine a scanning path for forming a hinged flap, based on flap parameters, which may include one or a combination of a thickness of the flap, a centration of the flap, a position of a hinge of the flap, a side cut angle (measured relative to an optical axis of the laser optical system) and a size of the flap (such as a diameter of the flap).

The controller may be configured to generate the scanning pattern so that the laser pulses are overlapping or non-overlapping. A lateral displacement of neighboring laser pulses may be less than 30 micrometers, or less than 20 micrometers, or less than 10 micrometers. The displacement may be greater than 1 micrometer or greater than 2 micrometers.

The eye treatment system may be configured to scan the laser focus within the cornea so as to form a lamella, which is at least partially separated from the surrounding corneal tissue. The lamella may be a hinged flap. The hinge may be configured so that the flap can be folded back to expose underlying corneal tissue, which is covered by the non-folded flap. The exposed corneal surface can be targeted by an ablation laser beam, which is incident on the eye. The exposed corneal surface may be a stromal surface. A hinge of the hinged flap may be formed by a tissue portion through which the flap is unseparated from the surrounding tissue. An anterior surface of the hinged flap may be a portion of an anterior surface of the cornea.

Alternatively, the lamella may be a portion of the cornea that is completely isolated from the surrounding corneal tissue so that the isolated lamella can be removed from the eye to be replaced by a transplant. A further example for the lamella is an intracorneal lamella, which is completely located within the cornea. At least a portion of the intracorneal lamella may be in the form of a lenticule. The lenticule may have a shape representing a positive or negative optical power. By way of example, the intracorneal lamella may be formed for performing FLEx or SMILE procedures.

The lamella may be a corneal surface lamella. The term “corneal surface lamella” may be defined to mean that (a) at least a portion of an anterior surface of the lamella is a portion of the anterior surface of the cornea (such as in LASIK procedures) or that (b) at least a portion of a posterior surface of the lamella is a portion of a posterior surface of the cornea (such as in posterior lamellar keratoplasty procedures). The corneal surface lamella may be flap or may be completely isolated from the surrounding corneal tissue.

The term “contact surface” as used herein may be defined to mean surface portion of the contact element, which, during treatment, is in contact with the cornea. The eye treatment system may be configured so that a diameter of the contact surface is equal to or larger than 6 millimeters or equal to or larger than 8 millimeters.

The contact element may be releasably attachable or non-releasably attached to a fixation system (such as a suction ring), which is configured to be fixed to the patient, in particular to the eye. It is also conceivable that the contact element and the fixation system are formed as a single piece. The fixation system may be configured to be fixed to the eye using vacuum. Additionally or alternatively, the laser system may include a coupling mechanism for detachably coupling the contact element relative to the laser optical system. The coupling mechanism may be configured so that the contact element is directly or indirectly coupled to the laser optical system.

At least a portion of the contact element may be lens-shaped. At least a portion of the lens-shaped portion may be traversed by the laser beam. The lens-shaped portion of the contact element may have a positive or negative optical power or may be devoid of optical power. At least a portion of the contact element, in particular the lens-shaped portion of the contact element may be transparent or substantially transparent for the pulsed laser beam. The lens-shaped portion may further be transparent for light of a measuring arm of an optical coherence tomography (OCT) system of the eye treatment system. A center wavelength of the OCT measuring arm may be within a range of between 750 and 1400 nanometers. Additionally or alternatively, the lens-shaped portion may be transparent for a plurality of wavelengths, which are within the visible wavelength range, i.e. within a range of between 380 nanometers and 750 nanometers.

By way of example, the contact element or the lens-shaped portion may be at least partially made of a polymer, such as cyclo olefin polymer (COP) or Polymethylmethacrylate (PMMA). Alternatively, the contact element or the lens-shaped portion may be at least partially made of glass. At least a portion of the contact surface and/or at least a portion of the proximal surface of the lens-shaped portion may be coated.

The contact element, in particular the lens-shaped portion may be the only component, which is in the beam path of the laser beam between the laser optical system and the eye so that between the laser optical system and the lens-shaped portion, the laser beam passes through air or vacuum. Alternatively, one or more further optical elements may be in the beam path of the laser beam between the lens-shaped portion and the focusing optical system.

According to an embodiment, the scanning system includes an axial scanning system for scanning the laser focus along an axis of the laser beam. The scanning system may further include a beam deflection scanning system for scanning the laser beam through deflection of the laser beam. The axial scanning system may be in the beam path of the laser beam between the laser source and the beam deflection scanning system. The scanning system may provide three degrees of freedom for performing the three-dimensional scanning of the focus in the cornea. One of the three degrees of freedom may be provided by the axial scanning system. The remaining two degrees of freedom of the scanning system may be provided by a beam deflection scanning system.

The axial scanning system may be configured to axially scan the focus of the laser beam through varying an angle of divergence or convergence of the laser beam. The divergence or convergence may be measured at a location along the axis of the laser beam, where the laser beam exits from the axial scanning system. The terms “divergence” or “convergence” as used herein may be defined to mean an angular measure of the increase or decrease in beam diameter or radius with distance. In addition to a scanning movement of the laser focus along the axis of the laser beam, the axial scanning system may also cause a deflection of the laser beam so that the focus of the laser beam performs a lateral movement within the cornea concurrently with the axial movement. The lateral movement of the laser focus may be smaller than the axial movement of the laser focus.

A deflection of the laser beam, which is performed using the beam deflection scanning system may adjust a lateral position of the laser focus relative to an optical axis of the focusing optical system within the cornea.

According to a further embodiment, compared to a plane-parallel applanation plate, the contact element is configured to reduce a variation of a depth of at least a portion of a scanning plane of the laser focus. The depth may be measured relative to the anterior surface of the cornea. Additionally or alternatively, the scanning plane may correspond to a constant scanning state of the axial scanning system.

The reduction of the depth variation may be measured compared to a depth variation of a scanning plane within the cornea, when a plane-parallel applanation plate is used for applanating the cornea and the axial scanning system is in the same scanning state. The depth variation may be defined to be a maximum difference between depth values of the portion of the scanning plane.

The plane-parallel applanation plate may have two parallel opposing surfaces, which are traversed by the laser beam. The plane-parallel applanation plate may be in the beam path of the laser beam replacing the lens-shaped portion of the contact element. The opposing surfaces of the applanation plate may be oriented perpendicular to the optical axis of the laser optical system. The plane-parallel applanation plate may be made from glass for from a same or substantially same material as the lens-shaped portion of the contact element.

A thickness of the plane-parallel applanation plate may be equal to or less than 40 millimeters or equal to or less than 30 millimeters or equal to or less than 20 millimeters or equal to or less than 10 millimeters. The thickness may be equal to or greater than 0.5 millimeters or equal to or greater than 1 millimeter or equal to or greater than 10 millimeters. The plane-parallel applanation plate may be pressed against the eye so that the contact surface (i.e. the surface of the applanation plate, which is in contact with the anterior surface of the eye) has a same or substantially same extent, as compared to the contact element.

The reduction of the depth variation, caused by the contact element, may be generated at least for each point of the scanning plane having a distance from the optical axis, which is less than 2 millimeters or less than 4 millimeters, or less than 5.5 millimeters, or less than 6 millimeter.

The unreduced depth variation of the scanning plane (i.e. when the plane-parallel applanation plate is used) may be at least partially caused by a field curvature of the laser optical system, in particular a field curvature of the focusing optical system.

The scanning state of the axial scanning system may be defined to mean a configuration of the axial scanning system, which corresponds to an axial scanning position of the laser focus along the axis of the laser beam and/or a divergence or convergence of the laser beam at a location, where the laser beam exits from the axial scanning system.

According to a further embodiment, the contact element is configured so that the depth variation of the scanning plane is less than 30 micrometers, or less than 20 micrometers, or less than 10 micrometers, or less than 6 micrometers, or less than 2 micrometers, at least for each point having a distance from an optical axis of the focusing optical system which is less than 2 millimeters or less than 4 millimeters or less than 5.5 millimeters or less than 6 millimeters.

According to an embodiment, the contact element includes a proximal surface, which is in the beam path of the laser beam and opposite to the contact surface. At least a portion of the proximal surface may have a shape, which is convex toward the incident laser beam. Alternatively, at least a portion of the proximal surface may have a shape, which is concave toward the incident laser beam or which is planar. The planar shape may be perpendicular or substantially perpendicular relative to the optical axis of the laser optical system. The local radius of curvature of the convex or concave shape of the proximal surface may depend on the field curvature of the laser optical system. At least a portion of the convex shape may have a radius of curvature, which is greater than 10 millimeters or greater than 15 millimeters, or greater than 30 millimeters or greater than 50 millimeters, or greater than 100 millimeters or greater than 150 millimeters. The value of the radius of curvature of the proximal surface may depend on the optical design of the laser optical system.

Specifically, at least for each location on the proximal surface having a distance of less than 3 millimeters, or a distance of less than 4 millimeters or a distance of less than 6 millimeters from an apex of the proximal surface, a local radius of curvature of the proximal surface may be greater than 10 millimeters, or greater than 15 millimeters, or greater than 30 millimeters, or greater than 50 millimeters, or greater than 100 millimeters or greater than 150 millimeters.

The shape of the proximal surface portion may contribute to the reduction of the depth variation of the scanning plane. Additionally or alternatively, the shape of the proximal surface portion may reduce a lateral focus diameter of the laser focus, as compared to a planar shape of the proximal surface portion or as compared to the plane-parallel applanation plate. The focal spot diameter may be measured as an 80% encircled energy diameter.

According to an embodiment, the laser beam is one of a plurality of laser beams, which are generated by the laser optical system using a beam multiplier of the eye treatment system. The beam multiplier may be arranged so that the pulsed laser light impinges on the beam multiplier. The scanning system may be configured to scan an ordered or disordered one- or two-dimensional focus array within the cornea, which is generated by the laser optical system using the plurality of laser beams. The foci of the focus array may be synchronously scanned by the scanning system. Specifically, the foci may be scanned in time and spatial synchronization, wherein the scanning paths of the foci are laterally and/or axially displaced from each other.

The beam multiplier may include an ordered or disordered one-, two or three-dimensional array of lenses or lens positions, such as an array of microlenses. The lenses may be illuminated in parallel by the pulsed laser beam (i.e. the lenses are not traversed successively by the laser beam). The principal planes of the lenses of the array may be arranged or substantially arranged in a plane. Additionally or alternatively, the beam multiplier may include an ordered or disordered array of mirrors, in particular an array of micro-mirrors. The mirrors may be arranged in the beam path of the pulsed laser so that they are illuminated in parallel.

Additionally or alternatively, the beam multiplier may include a phase mask or a spatial light modulator (SLM). The SLM may be transmissive or reflective. The SLM may be an amplitude-only, a phase-only, or a phase-amplitude SLM.

The foci of the focus array may be located at a constant or substantially constant depth, as measured from an anterior surface of the cornea.

According to an embodiment, for each of the foci of the focus array, compared to a plane-parallel applanation plate, the contact element is configured to reduce a variation of a depth of at least a portion of a scanning plane of the respective focus and of the individual foci relative to each other. The depth may be measured relative to the anterior surface and the scanning plane and/or may correspond to a constant scanning state of the axial scanning system.

According to a further embodiment, for each of the foci of the focus array, the depth variation may be less than 30 micrometers, or less than 20 micrometers, or less than 10 micrometers, or less than 6 micrometers, or less than 2 micrometers, at least for each point having a distance from an optical axis of the laser optical system which is less than 2 millimeters or less than 4 millimeters, or less than 5.5 millimeters, or less than 6 millimeters.

According to a further embodiment the axial scanning system includes a first optical system, which has a negative optical power. The axial scanning system may further include a second optical system, which has a positive optical power. The second optical system may be in the beam path of the laser beam between the first optical system and the deflection scanning system. The axial scanning system may be configured so that a distance between the first optical system and the second optical system is controllably variable. The first and/or second optical system may include or consist of one or more lenses. The distance may be measured along an optical axis of the axial scanning system.

According to an embodiment, the axial scanning system includes one or more displaceable lenses, which are in the beam path of the laser beam. The axial scanning system may be configured so that the one or more displaceable lenses are controllably displaceable in a direction parallel or substantially parallel to an optical axis of the one or more displaceable lenses. The eye treatment system may include a controller, which is in signal communication with an actuator of the axial scanning system. The actuator may be configured to displace the one or more displaceable lenses based on signals received from the controller.

According to an embodiment, the eye treatment system is configured for forming a lamella of corneal tissue, in particular a corneal flap, an intracorneal lenticule or a corneal surface lamella. The laser system may include a controller, which is configured to control the laser optical system to scan the focus within the cornea to at least partially isolate the lamella from surrounding corneal tissue using a subsurface cut and a side cut. The subsurface cut may represent at least a portion of an anterior or posterior surface of the lamella. The subsurface cut may represent at least 50% or at least 80% of the anterior or posterior surface of the lamella. The subsurface cut may be formed at a constant or substantially constant scanning state of the axial scanning system. The side cut may extend or may substantially extend to the anterior surface of the cornea. By way of example, the side cut may extend into the epithelium without extending to the anterior surface of the cornea so that the side cut only substantially extends to the anterior surface. In alternative embodiments, the side cut substantially extends or extends to the posterior surface of the cornea.

At least a portion of an anterior surface of the lamella may be a portion of an anterior surface of the cornea. Alternatively, at least a portion of a posterior surface of the lamella may be a portion of a posterior surface of the cornea.

According to an embodiment, the beam deflection scanning system includes two or three scanning mirrors. Each of the scanning mirrors may be part of a galvanometer scanner, in particular a resonant galvanometer scanner of the scanning system. Each of the scanning mirrors may be rotatably supported. The rotation axes of at least two of the scanning mirrors may be oriented non-parallel relative to each other.

According to a further embodiment, the beam deflection scanning system includes three scanning mirrors. A first and a second one of the three scanning mirrors may be configured to provide one of the two angular scanning dimensions of the beam deflection system. The first and second scanning mirrors may be configured so that a beam deflection generated by the first and second scanning mirrors pivots the laser beam about a pivot point, which is in the beam path of the laser beam downstream of the two scanning mirrors. The pivot point may be located on a reflective surface of the third scanning mirror, in particular on a portion of the reflective surface, which is located on or substantially on a rotation axis of the third mirror.

According to a further embodiment, the laser system is configured so that the contact element is detachably coupleable relative to the laser optical system. The contact element may be the only optical element, which is in the beam path of the laser beam between the laser optical system and the eye.

The contact element may include or may be attached to a coupling portion for coupling the contact element to a corresponding coupling portion which is provided by the laser optical system or which is rigidly connected to the laser optical system. In alternative embodiments, the corresponding coupling portion is displaceably supported in a direction parallel to an optical axis of the laser optical system.

According to an embodiment, the laser optical system includes a beam combiner, which is in the beam path of the laser beam between the scanning system and the eye.

According to a further embodiment, the beam combiner is configured for combining the beam path of the laser beam with a beam path of an imaging system of the eye treatment system. The imaging system may include an image sensor. The image sensor may include a two-dimensional ordered or unordered array of pixels. The image sensor may be sensitive to one or more wavelengths within a range of between 380 nanometers and 950 nanometers or within a range of between 380 nanometers and 1400 nanometers. The imaging system may be configured to acquire a frontal image of the eye and/or a portion of the contact element. Additionally or alternatively, the imaging system may include an optical coherence tomography system. The optical coherence tomography system may be configured to acquire a cross-sectional image of the cornea and/or at least a portion of the natural lens of the eye.

According to a further embodiment, the beam combiner may be configured to deflect the pulsed laser beam in a direction toward or substantially toward the eye. The beam combiner may include a mirror and/or a prism. The beam combiner may be configured as a dichroic beam combiner. The beam combiner may be in the beam path of the laser beam downstream of the focusing optical system, within the focusing optical system or upstream of the focusing optical system. Upstream of the beam combiner, the beam path of the laser may extend in a horizontal direction or may substantially extend in a horizontal direction. Downstream of the beam combiner the laser beam may extend or substantially extend in a vertical direction.

According to a further embodiment, at least a portion of the convex shape of the contact surface has a radius of, curvature, which is greater than 10 millimeters, or greater than 50 millimeters or greater than 100 millimeters or greater than 150 millimeters. Additionally or alternatively, at least a portion of the convex shape of the contact surface has a radius of curvature, which is less than 500 millimeters, or less than 300 millimeters, or less than 250 millimeters or less than 200 millimeters. The radius of curvature may be a local radius of curvature. Specifically, the radius of curvature may vary over the convex shape of the contact surface.

According to an embodiment, the contact surface has a convex shape at least for each point having a distance of less than 2 millimeters, or less than 4 millimeters, or less than 6 millimeters, as measured from an apex of the contact surface.

A lateral extent of the convex shape of the contact surface may correspond or substantially corresponds to or is larger than a lateral extent of the posterior or anterior surface of the lamella.

According to a further embodiment, at least for each location on the contact surface having a distance of less than 3 millimeters, or less than 4 millimeters or less than 6 millimeters from an apex of the contact surface, a local radius of curvature of the contact surface is greater than 10 millimeters, or greater than 50 millimeters or greater than 100 millimeters or greater than 150 millimeters. Additionally or alternatively, the local radius of curvature may be less than 500 millimeters, or less than 300 millimeters, or less than 250 millimeters or less than 200 millimeters.

According to a further embodiment, the laser system is configured so that the contact element is detachably coupleable relative to the laser optical system. In the coupled state, the contact element may be in a predefined radial position and/or a predefined inclination relative to an optical axis of the focusing optical system. By way of example, in the coupled state, the contact element may be displaceably supported in a direction parallel to the optical axis of the laser optical system. Alternatively, in the coupled state, the contact element may be not only in a predefined radial position but in a predefined three-dimensional position relative to the laser optical system. By way of example, the contact element may be in rigid connection with (or may be formed in a single piece with) a coupling portion, which is configured for engagement with a corresponding coupling portion provided of the laser optical system.

According to a further embodiment, the laser optical system is configured to generate, using the laser beam, a substantially laterally extending subsurface cut within the cornea. The convex shape may be configured so that gas, which is caused by the formation of the substantially laterally extending subsurface cut, is guided substantially in a direction away from an apex of the convex shape and/or from an optical axis of the laser optical system. The optical axis of the laser optical system may extend through the laterally extending subsurface cut. The subsurface cut may be located at a constant or non-constant depth relative to the anterior surface. At a location, where the subsurface cut intersects the optical axis, the subsurface cut may be oriented perpendicular or substantially perpendicular relative to the optical axis.

According a further embodiment, the eye treatment system includes a controller, which is configured to control the laser optical system to scan the focus within the cornea to at least partially isolate the lamella from surrounding corneal tissue using a subsurface cut and a side cut. The subsurface cut may represent at least a portion of an anterior or posterior surface of the lamella and the side cut may represent at least a portion of a rim of the lamella. For forming the lamella, the controller may be configured to control the laser optical system to: form one or more gas conducting gas release cuts in the cornea, wherein for each of the gas release cuts, the respective cut extends to an anterior or posterior surface of the cornea and at least a portion of the gas release cut forms at least a portion of the rim of the lamella; to form at least a portion of the subsurface cut after formation of the one or more gas release cuts; and to complete the side cut after formation of the subsurface cut so that for each of the gas release cuts, at least a portion of the respective gas release cut forms a portion of the side cut.

At least a portion or all of the subsurface cuts, the side cut and/or the gas release cut may be perforated or continuous. The term “perforated cut” as used herein may be defined to mean a cut, which includes a plurality of bridges of corneal tissue, which connect two mutually opposing surfaces, which are separated by the cut. The term “continuous cut” as used herein may be defined to mean a cut, which is free or substantially free from bridges, i.e. it is non-perforated.

The side cut and/or the rim may at least partially surround an optical axis of the eye and/or the optical axis of the laser optical system. The side cut and/or the rim may be circumferentially open or closed. At least a portion of the side cut and/or the rim may extend or may substantially extend to the anterior surface of the cornea. By way of example, at least a portion of the side cut may extend into the epithelium without extending to the anterior surface of the cornea so that the side cut only substantially extends to the anterior surface. The rim and/or the side cut may connect the anterior surface of the lamella with the posterior surface of the lamella.

At least a portion of an anterior surface of the lamella may be a portion of an anterior surface of the cornea. Alternatively, at least a portion of a posterior surface of the lamella may be a portion of a posterior surface of the cornea.

The optical axis of the eye and/or the optical axis of the laser optical system may extend through the subsurface cut. The term “optical axis of the eye” as used herein may be defined to be an axis which connects a center of the pupil with a center of curvature of the cornea. The side cut may be circumferentially open to define a hinge of the hinged flap. A circumferential extent of the hinge (i.e. the circumferential region, where the side cut is open) may be greater than 10 degrees or greater than 30 degrees. The circumferential extent may be less than 120 degrees or less than 90 degrees, or less than 70 degrees. A minimum diameter of the side cut may be greater than 4 millimeters.

The one or more gas release cuts may be configured to release gas, which is caused by photodisruptions, in particular by laser-induced optical breakdown in a focal region of the laser beam. The gas may be guided by the gas release cut from the subsurface cut to the anterior or posterior surface of the cornea. A local pressing force per unit area p/A, which is generated on the anterior surface of the cornea using the convex shape of the contact surface and which decreases with increasing radial distance r from the apex of the convex contact surface further causes the gas to be guided through the gas release cuts to the anterior or posterior surface of the cornea.

Completing the side cut may include connecting two or more of the gas release cuts. More than two gas release cuts may be connected in series. For each of the gas release cuts, at least a portion of the respective gas release cut may represent a circumferential portion of the side cut.

According to a further embodiment, after completion of the side cut, for each gas release cut, which was used for releasing gas from the subsurface cut, at least a portion of the respective gas release cut is part of the side cut. Specifically, each of the gas release cuts may completely form part of the side cut.

According to a further embodiment, the lamella is a hinged flap and the side cut is the rim of the hinged flap. The side cut may extent to the anterior surface of the cornea along a full circumference of the side cut, wherein a circumference of the side cut is open or closed. In an embodiment, in which at least a portion the posterior surface of the lamella is a portion of the posterior surface of the cornea, the side cut may extend to the posterior surface of the cornea along a full circumference of the side cut, wherein circumference of the side cut is open or closed.

According to a further embodiment, the side cut is connected to the subsurface cut at a peripheral portion of the subsurface cut.

According to a further embodiment, a combined circumferential length of the one or more gas release cuts, as measured along a circumference of the side cut, amounts to less than 60% or less than 50% of a circumferential length of the side cut. If the side cut is circumferentially open, this may reduce its circumference. A circumferentially open side cut may be due to the fact that the side cut does not extend through a tissue portion, which forms the hinge.

According to a further embodiment, at least one or each of the one or more of the gas release cuts has a circumferential extent, which is greater than 5 degrees or greater than 10 degrees. The circumferential extent may be less than 120 degrees, or less than 90 degrees, or less than 60 degrees or less than 45 degrees or less than 20 degrees.

According to a further embodiment, forming the subsurface cut includes automatically or interactively selecting one of a plurality of pre-defined scanning patterns for forming the subsurface cut. The controller may further be configured to determine, based on the selected scanning pattern, which is used for forming the subsurface cut: i) for at least one of the gas release cuts, a parameter of a) a geometric shape, b) a position and/or c) an orientation and/or ii) a number of the one or more gas release cuts. The geometric shape may be an intrinsic geometric shape of the gas release cuts, such as curved or plane.

The interactive selection of the scanning pattern may include receiving, via a user interface of the controller, user input, indicative of one or more parameters of a scanning pattern and/or one of a plurality of pre-defined scanning pattern types. Each of the pre-defined scanning pattern types may be represented by one or more data structures. The data structures may be configurable using the one or more parameters. By way of example, the pre-defined scanning pattern types may include the scanning pattern type “meandering scanning pattern” and the scanning pattern type “spiral scanning pattern”. The one or more parameters of the scanning pattern may, for example, include a parameter of a line-to-line spacing of a meandering scanning pattern and/or a starting point of the scanning pattern.

According to a further embodiment, the controller is configured to control the laser optical system to form at least one of the gas release cuts so that the gas release cut contacts the subsurface cut at a location, which is scanned by an initial section of the scanning path, which corresponds to less than 60% or less than 50%, or less than 30%, or less than 20% of the total scanning path for forming the subsurface cut.

According to a further embodiment, each of the gas release cuts has a peripheral extent, which is less than 40 degrees or less than 20 degrees or less than 10 degrees and a number of the gas release cuts may be larger than 5 or larger than 10 or larger than 20 or larger than 50.

According to a further embodiment, each of the gas release cuts opens into an anterior surface of the cornea at a location where the contact surface contacts the cornea.

Embodiments of the present disclosure pertain to a method of treating an eye for forming a lamella of corneal tissue, in particular a corneal flap, an intracorneal lamella or a corneal surface lamella. The method is performed using a laser optical system having a laser source configured to generate a pulsed laser beam having a pulse duration of less than 1 picoseconds and a scanning system for scanning a focus of the laser beam within a cornea to at least partially isolate the lamella from surrounding corneal tissue using a subsurface cut and a side cut. The subsurface cut represents at least a portion of an anterior or posterior surface of the lamella and the side cut represents at least a portion of a rim of the lamella. The method comprises forming, using the laser optical system, one or more gas conducting gas release cuts in the cornea, wherein for each of the gas release cuts, the respective cut extends to an anterior or posterior surface of the cornea. The method further comprises forming, using the laser optical system, at least a portion of the subsurface cut after formation of the one or more gas release cuts. The method further comprises completing, using the laser optical system, the side cut, after formation of the subsurface cut so that for each of the gas release cuts, at least a portion of the respective gas release cut forms a portion of the side cut.

### BRIEF DESCRIPTION OF THE DRAWINGS

FIG. 1 schematically illustrates a system according to a first exemplary embodiment for treating a cornea of a human eye;

FIG. 2 is are further schematic illustration of the laser system according to the first exemplary embodiment, which is shown in FIG. 1;

FIG. 3 A is a schematic illustration of a contact element, a suction ring and a coupling portion of the eye treatment system according to the first exemplary embodiment, which is illustrated in FIGS. 1 and 2;

FIG. 3B is a cross-sectional view of the contact element of the eye treatment system according to the first exemplary embodiment;

FIG. 4A is a cross-sectional view of a lens-shaped portion of the contact element of the eye treatment system according to the first exemplary embodiment in a state in which the lens-shaped portion is in contact with the cornea;

FIG. 4B is a graph showing curves, which illustrate depth variations of different scanning planes of the laser optical system of the first exemplary embodiment, if a plane-parallel applanation plate is used for contacting the cornea;

FIG. 4C is a graph showing curves, which illustrate depth variations of different scanning planes of the laser optical system of the first exemplary embodiment, if a the contact element is used for contacting the cornea;

FIG. 5A is a schematic illustration of an axial scanning system of the eye treatment system release system according to the first exemplary embodiment;

FIG. 5B is an schematic illustration of a beam deflection scanning system of the eye treatment system according to the first exemplary embodiment;

FIG. 5C is a schematic illustration of a beam combiner, a focusing optical system, the contact element, and imaging systems of the eye treatment system according to the first exemplary embodiment;

FIG. 6A is a schematic illustration of how gas bubbles, which are generated during formation of a subsurface cut using the eye treatment system according to the first exemplary embodiment are guided away from an optical axis of the eye treatment system;

FIG. 6B is a schematic illustration of a radial dependency of a force per area exerted on the anterior surface of the cornea using the contact element of the eye treatment system according to the first exemplary embodiment;

FIG. 7 is a schematic illustration of intracorneal pockets, which are used to receive the gas bubbles, which are guided away from the optical axis using the contact element of the eye treatment system according to the first exemplary embodiment;

FIGS. 8A and 8B are schematic illustration of gas release cuts, which are used to release the gas bubbles to the exterior, wherein the gas bubbles are guided away from the optical axis using the contact element of the eye treatment system according to the first exemplary embodiment;

FIGS. 9A to 12 are schematic illustrations of different scanning patterns, which are used to generate the subsurface cut by the eye treatment system according to the exemplary embodiment.

FIG. 13 is a schematic illustration of an eye treatment system according to a second exemplary embodiment.

### DETAILED DESCRIPTION OF EXEMPLARY EMBODIMENTS

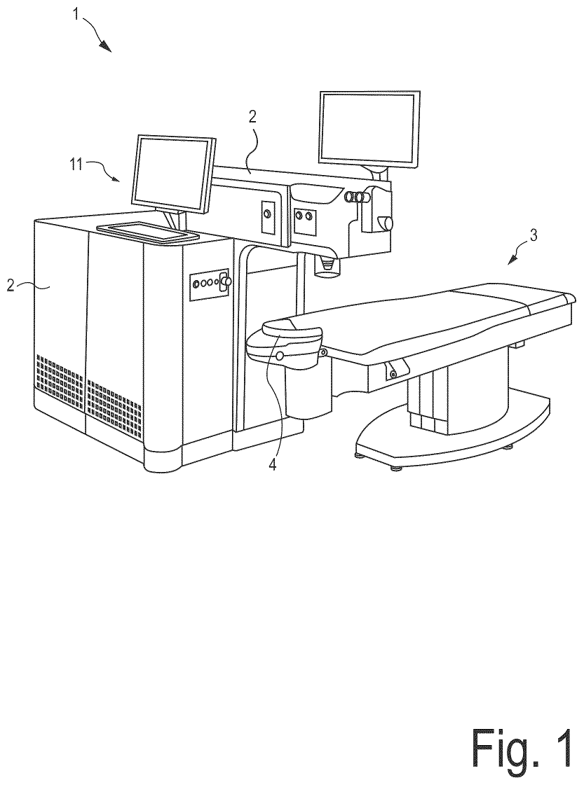

FIG. 1 is a schematic illustration of an eye treatment system 1 for performing laser surgery on an eye according to an exemplary embodiment. The system 1 includes a laser source and a laser optical system that are mounted within a housing 2. The laser optical system is configured to direct a laser beam to the eye of a patient who is disposed on a patient support structure 3 having a headrest 4 on which the patient's head is supported. In the exemplary embodiment, the laser source is configured to emit a pulsed laser beam having a pulse energy and a pulse duration, which is sufficient to generate laser-induced optical breakdown (LIOB) within the cornea of the patient's eye. The laser-induced optical breakdown generated by a laser pulse leads to photodisruption, so that a series of consecutive overlapping or closely located laser pulses generate cuts within the corneal tissue at locations. Photodisruption is a nonthermal photodisruption process, which separates stromal layers within the cornea. The laser optical system includes a scanning system, which is configured to scan a focus of the pulsed laser beam within the cornea to form perforated cuts or continuous (i.e. non-perforated) cuts. The perforated cuts may be generated by applying non-overlapping laser pulses, whereas the solid cuts may be generated using overlapping laser pulses.

In the exemplary embodiment, which is illustrated in FIG. 1, the laser optical system is configured so that the laser beam, which is incident on the patient's eye has a pulse duration of less than 1 ps or less than 800 femtoseconds. The pulse duration may be greater than 1 femtosecond or greater than 20 femtoseconds.

Additionally or alternatively, it is also conceivable that the laser system of the exemplary embodiment is configured so that laser pulses are applied to the cornea having an energy, which is below the threshold for generating LIOB. By way of example, a plurality of laser pulses, which have a pulse energy below the threshold for generating LIOB may be overlapped in a manner so as to cause tissue separation.

The corneal cuts can be used for performing a variety of different laser surgical procedures. By way of example, for performing laser-assisted in-situ keratomileusis (LASIK), the laser system may be configured for forming cuts, which partially isolate a corneal lamella so that the lamella forms a superficial flap. The flap is connected to the cornea via a tissue portion, which functions as a hinge. Using the hinge, the flap can be folded back to expose a surface of stromal tissue. An excimer laser (which can be part of the laser system illustrated in FIG. 1 or can be implemented in a separate laser system) is then scanned over the exposed stromal surface in a pattern that is calculated to ablate stromal tissue in a manner to at least partially correct a refractive error of the eye.

In a further example, the cuts form a subsurface intracorneal lenticule within the cornea, which is completely isolated from the surrounding tissue. The lenticule is shaped in a manner so that its extraction at least partially corrects for the refractive error of the eye. The extraction of the lenticule can, for example, be performed through a small incision, such as in “Small Incision Lenticule Extraction” (SMILE) procedures, or through a LASIK type hinged flap, such as in “Femtosecond Lenticule Extraction” (FLEx) procedures. Still further examples for laser surgery procedures in which the laser system of FIG. 1 can be used are lamellar corneal transplant techniques.

It has been shown that it is advantageous that the cuts within the corneal tissue are formed sufficiently fast so that the surgical procedure can be performed within a short period of time. Thereby, the surgery is less stressful for the patient. Further, this reduces the occurrence of errors caused by excessive movement of the patient.

On the other hand, as scan rates increase, greater demands are placed on the laser scanner used to direct the laser beam. Specifically, laser scanners, which are used to control the scanning motion may begin to introduce mechanical lag errors in focal point positioning at increased scan rates.

However, as is explained in more detail further below, the present inventors have demonstrated that it is possible to significantly reduce the time required for forming corneal cuts using femtosecond lasers. It has also been shown that it is possible to form cuts having a comparatively high degree of smoothness, which improves the visual results and the self-healing process after the surgery.

A further effect, which may lead to complications is the occurrence of gas bubbles, which are caused by laser-induced optical breakdown of corneal tissue in the focus of the laser beam. The vaporized tissue forms cavitation bubbles, which collapse and leave behind gas bubbles, which consist of carbon dioxide (CO₂), nitrogen (N₂) and water (H₂O) as main constituents. When the gas bubbles are trapped within the cornea, an opaque bubble layer (OBL) may be generated. Excessive opaque bubble layers can lead to interference in many stages of the surgical procedure such as flap creation, residual stroma bed measurements and eye tracking for positioning the excimer laser in LASIK procedures.

If there is an abnormality in the anterior cornea, such as an abnormality in the Bowman's membrane, it is possible that the gas bubbles migrate vertically and dissect superiorly toward Bowman's membrane and through the epithelium (vertical gas breakthrough). This can lead to scarring in LASIK surgeries so that the procedure should be terminated. The gas bubbles also can migrate into the anterior chamber, where they can interfere with the eye tracker of the excimer laser, which is used for ablation in LASIK surgical procedures.

However, as is described in detail further below, the present inventors have demonstrated that it is possible to efficiently remove the gas bubbles from subsurface cuts which are used to form the corneal lamella so that the above-described complications can be efficiently minimized.

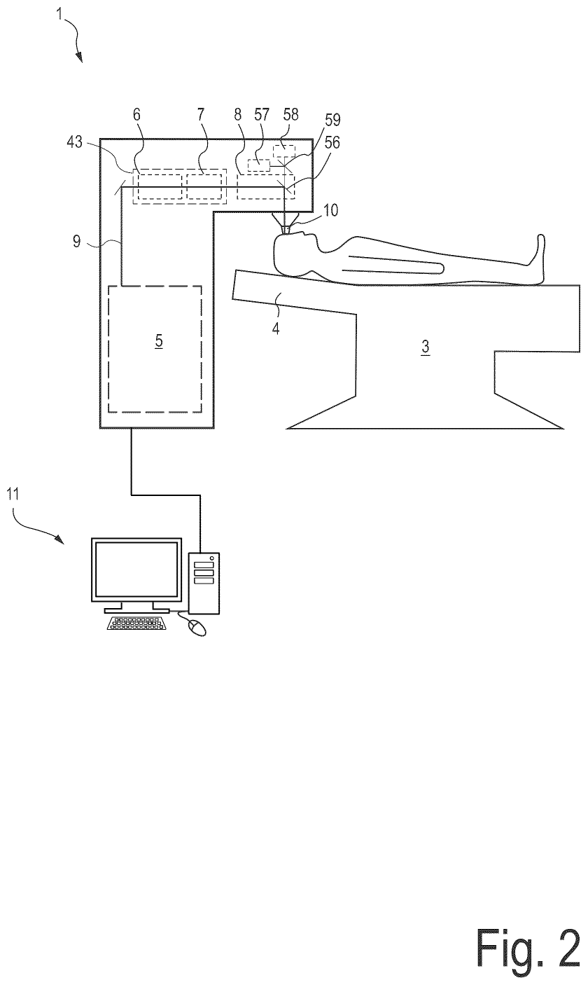

FIG. 2 is a further schematic illustration of the laser system according to the exemplary embodiment, which is illustrated in FIG. 1. The laser source 5 generates a laser beam 9, which traverses a scanning system 43, which includes an axial scanning system 6 and a beam deflection scanning system 7. The axial scanning system 6 is configured to scan the focus of the laser beam within the eye in a direction along a beam axis of the laser beam. The beam deflection scanning system 7 is configured to angularly deflect the laser beam in two angular dimensions.

Therefore, the axial scanning system and the beam deflection scanning system together provide a scanning system having three degrees of freedom so that the laser focus can be scanned in three dimensions within the eye. In the eye treatment system according to the exemplary embodiment, the deflection beam scanning system 7 is disposed in the beam path downstream of the axial scanning system 6. However, it is noted that the present disclosure is not limited to such a configuration and it is also conceivable that the beam deflection scanning system 7 is disposed in the beam path between the laser source 5 and the axial scanning system 6.

In the exemplary embodiment, which is shown in FIG. 2, the section of the laser beam, which exits from the beam deflection scanning system 7 is incident on a focusing optical system 8. The laser beam 9 traverses the focusing optical system 8 and a contact element 10, which is directly in contact with the anterior surface of the patient's cornea. As is further illustrated in FIG. 2, the eye treatment system further includes a controller 11 for controlling the laser source 5 and the laser optical system 43 to controllably scan the laser focus of the laser beam 9 within the cornea of the patient's eye.

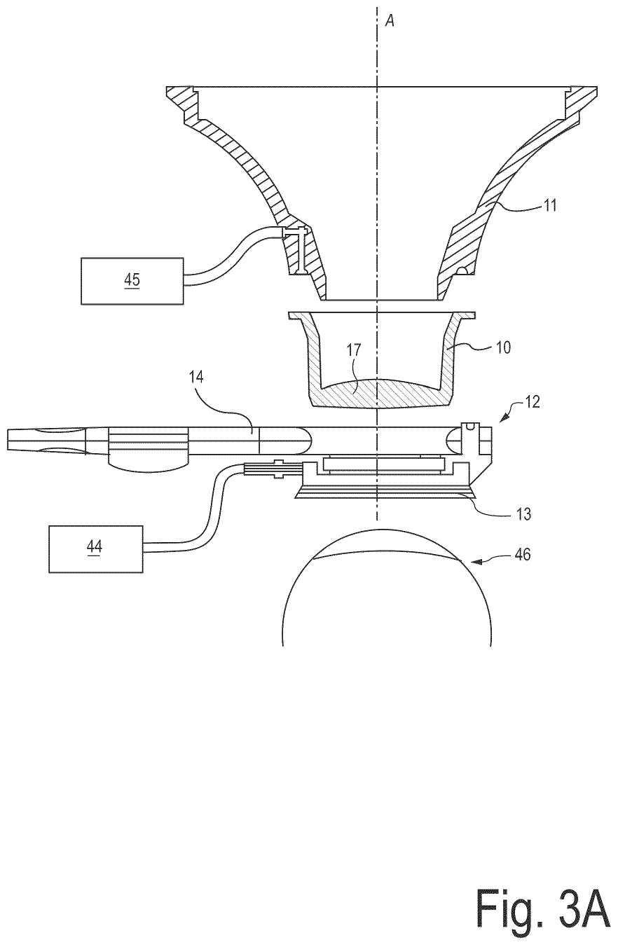

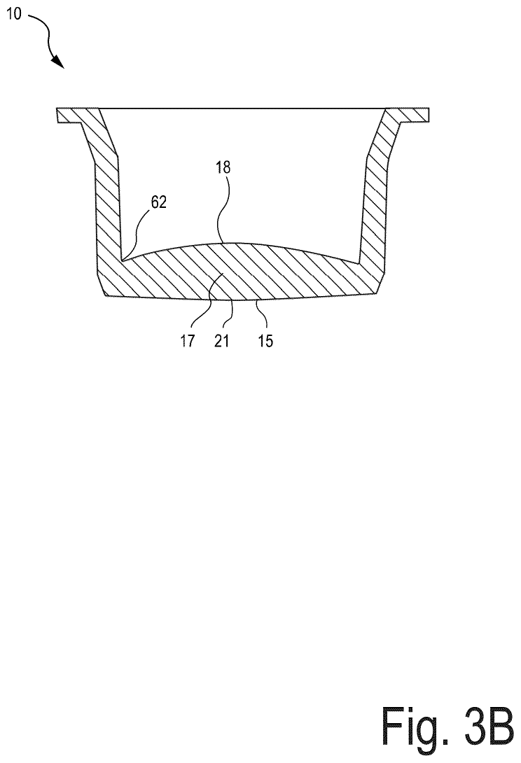

FIG. 3A schematically illustrates—in an exploded view—the contact element 10 of the eye treatment system of the exemplary embodiment and further components, which are used for coupling the contact element 10 relative to the laser optical system on the one hand and relative to the patient's eye 46 on the other hand. The eye treatment system 1 includes a coupling portion 11, which may be in rigid connection with the laser optical system or which may be displaceably supported in a direction parallel to the optical axis of the laser optical system. The contact element 10 and the coupling portion 11 are configured so that the contact element 10 is detachably coupleable to the coupling portion 11. In the coupled state, the contact element 10 may be in a substantially predefined position relative to the laser optical system and a pre-defined inclination relative to the optical axis A of the laser optical system. Alternatively, in the embodiment, in which the contact element 10 is displaceably supported in a direction parallel to the optical axis of the laser optical system, in the coupled state, the contact element 10 is in a pre-defined radial position relative to the optical axis and has a pre-defined inclination relative to the optical axis A. The contact element 10 is attached to the coupling portion 11 using a suction mechanism, which includes a suction source 45.

The eye treatment system 1 further includes a suction ring 12 which can be secured to the eye 46 and to which the contact element 10 is rigidly attachable. The suction ring 12 includes a skirt that forms a groove, which defines a suction channel between the skirt and an anterior surface of the eye 46. Generation of a vacuum in the vacuum passage using a vacuum source 44 therefore fixedly attaches the the suction ring 12 to the anterior surface of the eye 46.

The suction ring 12 is rigidly attached to a clamp mechanism 14 or formed with the clamp mechanism 14 as a single piece. The clamp mechanism 14 is used for securing the contact element 10 to the suction ring 12. One example for such a clamp mechanism 14 is disclosed in document US 2007/0093795 A1, the contents of which is incorporated herein by reference for all purposes. However, the present invention is not limited to configurations in which the contact element 10 is secured to the suction ring 12 using a clamp mechanism. Specifically, it is conceivable that the contact element 10 and the suction ring 12 are integrally formed, such as formed as a single piece or integrated into a one-piece assembly.

In the exemplary embodiment, the contact element 10 is integrally formed as a single piece or a one-piece assembly. However, it is also possible that the contact element 10 is made from multiple separable pieces. In the exemplary embodiment, the contact element 10 has a lens-shaped portion 17, which is made from material which is transparent or substantially transparent to laser light of the pulsed laser beam. By way of example, the patient interface is made of Cyclo Olefin Polymer (COP). However, it is also conceivable that the contact element is made from a different material, such as Polymethylmethacrylate (PMMA), glass, Makrolon, polyester or polycarbonate.

FIG. 3B is an enlarged cross-sectional view of the contact element 10. The lens-shaped portion 17, of the contact element 10 includes a contact surface 15, for contacting the anterior surface of the cornea. The contact surface 15 has a shape, which is convex toward the eye. After the contact element 10 has been attached to the eye 46 using the suction ring 12, the convex shape of the contact surface 15 deforms the anterior surface of the cornea so that a portion of the anterior surface is concave.

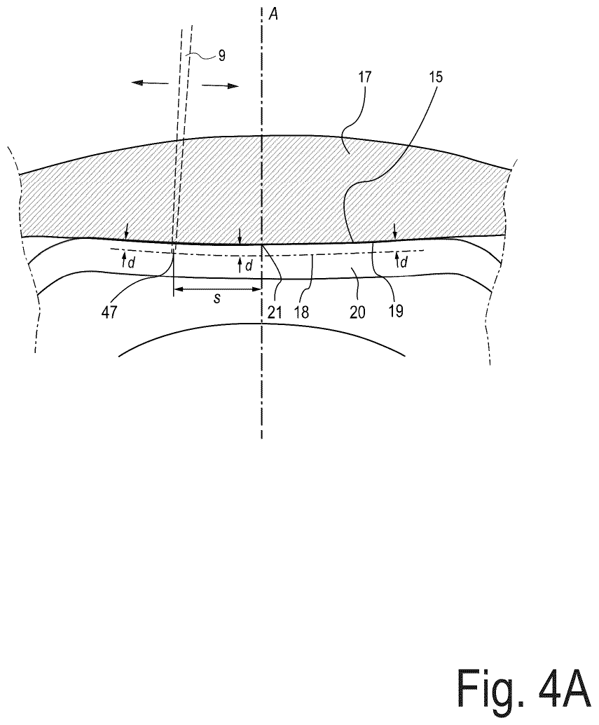

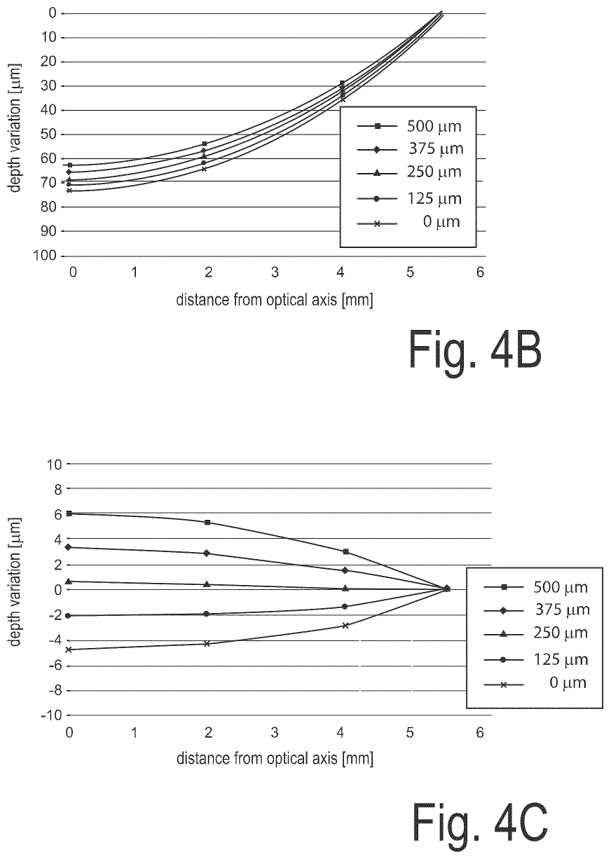

It has been demonstrated by the inventors (and is explained in more detail in connection with FIG. 4A) that the convex shape allows forming the contact surface 15 and the proximal surface 18 of the lens-shaped portion 17 so that subsurface cuts can be generated in the cornea, which have a constant or substantially constant depth from the anterior surface without changing the scanning state of the axial scanning system. In other words, it is possible to form subsurface cuts only by using the beam deflection scanning system (designated with reference numeral 7 in FIG. 2) and without changing the axial position of the laser focus using the axial scanning system (designated with reference numeral 6 in FIG. 2).

FIG. 4A schematically illustrates a cross-sectional view which is taken along an optical axis A of the eye and which shows a cross-section of the lens-shaped portion 17 of the contact element in a state in which the lens-shaped portion 17 is in contact with the cornea 20 of the eye. The laterally extending dashed-dotted line (designated with reference numeral 18) schematically illustrates a cross-section through a curved plane, which represents locations of the laser focus of the laser beam 9 for a constant scanning state of the axial scanning system and for different deflection angles of the beam deflection system. The curved plane therefore represents a scanning plane 18, which corresponds to a constant scanning state of the axial scanning system.

The reason for the fact that a constant state of the axial scanning system generates a curved scanning plane 18 within the cornea 20 is that the laser optical system, in particular the focusing optical system, has a field curvature so that for a constant scanning state of the axial scanning system and different deflection angles of the beam deflecting scanning system, the laser focus is within a curved plane within the cornea 20.

The inventors have demonstrated that it is possible to configure the lens-shaped portion 17 of the contact element so that the contact element reduces a variation of a depth of at least a portion of the scanning plane 18, wherein the depth is measured relative to the anterior surface 19 of the cornea 20. Therefore, the portion of the scanning plane 18 is located at a constant or substantially constant depth d, as measured from the anterior surface 19 of the cornea 20.

Using the lens-shaped portion 17 of the contact element for at least partially reducing the variation of the depth of at least a portion of the scanning plane 18 has the advantage that it is not necessary to provide additional correction optics in the laser optical system or in the beam path of the laser beam between the laser optical system and the lens-shaped portion 17 of the contact element.

The lens-shaped portion 17 of the contact element may be configured so that a depth variation of the scanning plane 18 is less than 30 micrometers, or less than 20 micrometers, or less than 10 micrometers, or less than 6 micrometers or less than 2 micrometers, at least for each point having a distance s from the optical axis A of the laser optical system of less than 2 millimeters or less than 4 millimeters or less than 5.5 millimeters or less than 6 millimeters.

Such an accuracy allows formation of flaps having a constant intended thickness. Specifically, the intended thickness may be greater than 70 micrometers or greater than 100 micrometers. The intended thickness may be less than 170 micrometers or less than 150 micrometers. The thickness of the formed flap may be within a range of +/−10% or within a range of +/−5% of the intended flap thickness.

Specifically, the inventors have demonstrated that when designing the laser optical system and the lens-shaped portion, a radius of curvature of a starting surface of the contact surface can be chosen so that it is substantially equal to or is equal to a radius of curvature of the scanning plane when the lens-shaped portion is replaced by a plane-parallel applanation plate. The laser optical system and the lens-shaped portion can then be optimized using a boundary condition, which sets a limit for the depth variation of the scanning plane, measured relative to the anterior surface of the cornea by means of optical calculation. The optimization may be an optimization of one or more parameters. One of the one or more optimized parameters may be or may depend on the focal diameter of the laser beam in the cornea.

It has further been shown that the arrangement of the scanning system 43 (shown in FIG. 2), which includes the axial scanning system 6 and the beam deflection scanning system 7 between the laser source 5 on the one hand and the focusing optical system 8 on the other hand reduces the required curvature (i.e. it increases the radii of curvature) of the convex shape of the contact surface 15 (shown in FIG. 4) so that an increase in intraocular pressure, which is generated by the convex shape of the contact surface 15 is kept within a range, which is acceptable for the patient. It has further been shown that due to the reduced required curvature, a remaining depth variation after optimization of the convex shape of the contact surface is smaller.

By way of example, and referring to FIG. 3B, at least for each location on the contact surface 15 having a distance of less than 3 millimeters, or a distance of less than 4 millimeters or a distance of less than 6 millimeters from an apex 21 of the contact surface 15, a local radius of curvature of the contact surface 15 is greater than 10 millimeters, or greater than 15 millimeters or greater than 30 millimeters or greater than 50 millimeters, or greater than 100 millimeters, or greater than 150 millimeters.

The apex 21 may be defined as the most distal portion of the convex portion of the contact surface 15, as seen along the optical axis of the laser optical system, which is in the beam path upstream of the lens-shaped portion. In a state in which the contact element 10 is coupled relative to the laser optical system, the optical axis of the laser optical system (designated with reference numeral A in FIG. 4A) may pass through the apex 21.

If the scanning state of the axial scanning system is constant over the time period during which the subsurface cut is formed, the cut can be formed within a comparatively short period of time. Generally, shorter surgical times are less stressful for the patient, and can reduce the likelihood of errors introduced by excessive movement of the patient. Also, shape irregularities of the cut, which may be introduced by movements of the laser focus along the beam axis using the axial scanning system are avoided. The convex shape of the contact surface therefore creates a smooth cut, which may improve the visual results.

Further, due to the fact that the scanning process is time efficient since no or only minor adaptations of the axial scanning system are necessary, it is possible to use meandering scanning patterns or raster scanning patterns, which otherwise would be much less time efficient, but which allow generation of a much smoother subsurface cut. In other words, the constant scanning state of the axial scanning system allows for faster scanning speeds also for meandering scanning patterns and raster scanning patterns and therefore eliminates the otherwise required reduction of treatment speed as compared to spiral or circular treatment patterns.

The contact element according to the exemplary embodiment can also be advantageously used for forming laterally extending subsurface cuts, which do not have a constant depth but a comparatively small deviation from a constant depth. Such subsurface cuts can, for example be used in FLEx or SMILE surgical procedures, when only minor refractive correction is required. It has been demonstrated by the inventors that also such cuts can be formed at a comparatively high speed, since the number and/or the extent of the adjustments of the axial scanning system for adjusting the position of the focus along the axis of the laser beam are comparatively small.

The graph of FIG. 4B shows curves, which illustrate depth variations of scanning planes of the laser optical system of the first exemplary embodiment, in a configuration in which a plane-parallel applanation plate is used in place of the lens-shaped portion for contacting the cornea. As is indicated in the legend of the graph of FIG. 4B, each of the curves represents a different scanning state of the axial scanning system so that each of the curves represents a different depth of the scanning plane measured relative to the anterior surface of the cornea and along the optical axis. The x-axis indicates the distance from the optical axis of the laser optical system. The curves are offset along the y-axis to facilitate comparison between them.

The graph of FIG. 4C shows curves, which illustrate depth variations of scanning planes of the laser optical system of the first exemplary embodiment, if the lens-shaped portion of the contact element is used for contacting the cornea. As can be seen by comparing the curves of FIG. 4B with the corresponding curves of FIG. 4C, compared to the plane-parallel applanation plate, the lens-shaped portion of the contact element reduces the variation of the depth of the scanning plane. Specifically, even for a scanning plane, which is located at a depth of 500 micrometers (indicated by squares), the depth variation of the scanning plane is less than 6 micrometers for each point having a distance from the optical axis of less than 5.5 millimeters.

Therefore, it has been shown that by using the contact element having the lens-shaped portion, it is possible to correct the field curvature for a wide variety of depths of the scanning plane. It has further been shown that the arrangement of the scanning system upstream of the focusing optical system together with the lens-shaped portion of the contact element having the convex contact surface allows an even more improved correction of the field curvature for a wider range of different depths. Therefore, it is possible to generate, for a wide range of different depth positions, subsurface cuts, which are located at a constant depth relative to the anterior surface of the cornea and which have a reduced depth variation. By way of example, in LASIK treatment procedures, this allows formation of flaps of different flap thicknesses, wherein each of the flaps has a more constant flap thickness and a more improved flap bed cut smoothness.

As has been explained in connection with FIG. 2, in the laser system of the exemplary embodiment, the beam deflection scanning system 7 is in the beam path of the laser beam 9 between the axial scanning system 6 and the focusing optical system 8. Therefore, the laser beam, which traverses the axial scanning system is undeflected (i.e. it extends along or substantially along the optical axis) so that the optical elements of the axial scanning system 6 can be configured to have a comparatively small effective diameter. This increases the scanning velocity of the axial scanning system, since the lenses which are displaced during the scanning process have a comparatively small mass. This can in particular be advantageous for forming the rim cut of the flap in LASIK surgical procedures, since forming the rim cut requires displacement of the laser focus along the beam axis to generate photodisruptions in different depths in order to connect the flap bed cut to the anterior surface of the cornea.

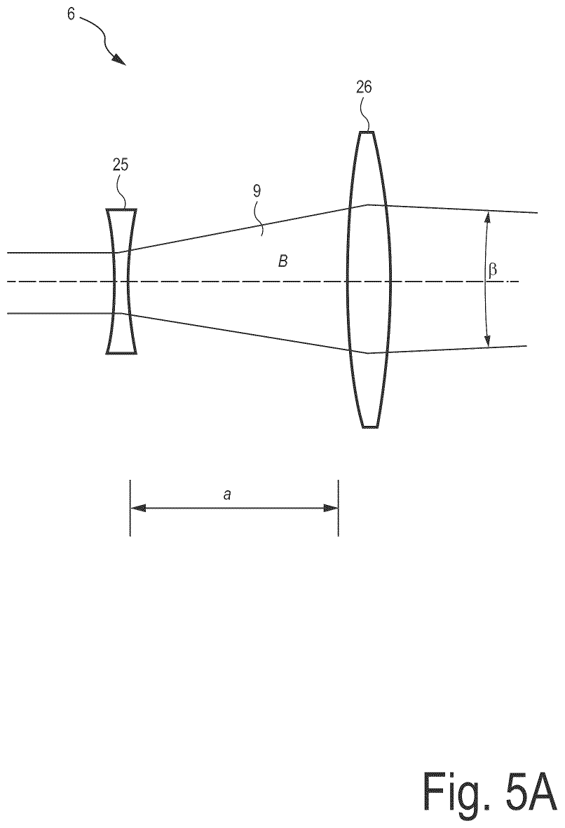

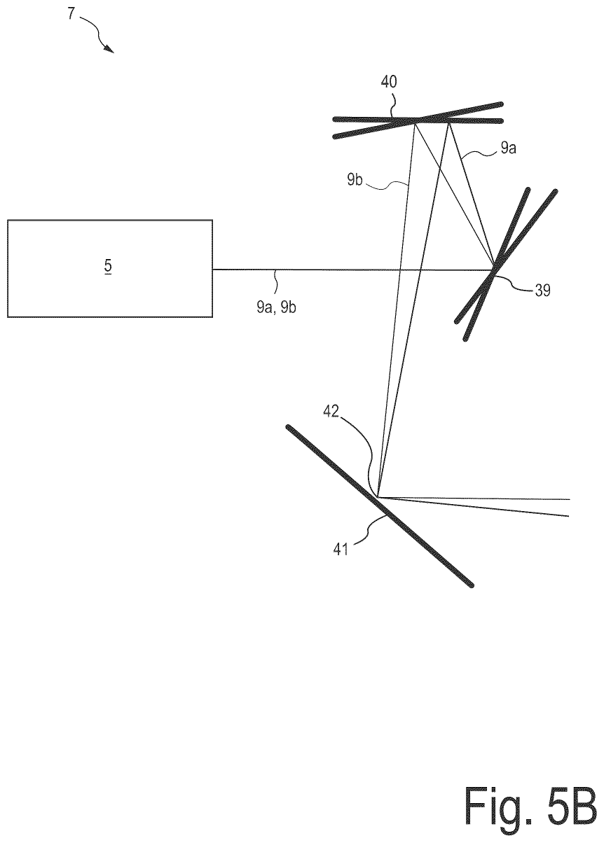

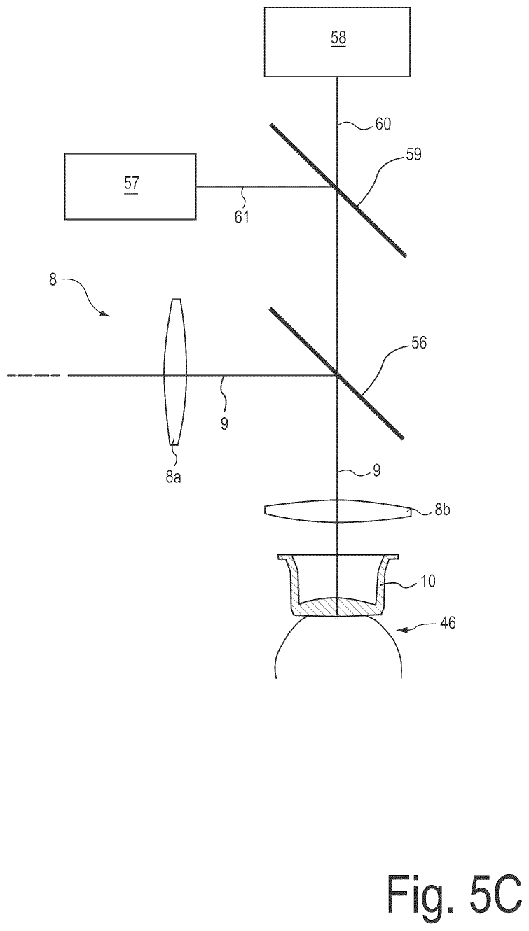

The configuration of the axial scanning system 6 is shown in FIG. 5A. The axial scanning system 6 includes a first optical system 25 (which includes a lens or lens assembly), which has a negative optical power. The section of the laser beam, which exits from the first optical system 25 is incident on the second optical system 26 (which includes a lens or lens assembly), which has a positive optical power. The axial scanning system 6 is configured so that displacing the focus along the beam axis includes varying a distance a between the first optical system 25 and the second optical system 26. By way of example, the first optical system 25 and/or the second optical system 26 are controllably displaceable along an optical axis B of the axial scanning system 6 using an actuator (not shown in FIG. 5A), which is in signal communication with the controller of the eye treatment system. The first and second optical systems 25, 26 are configured so that the variation of the distance a between the first and the second optical system 25, 26 varies an angle β of divergence or convergence of the section of the laser beam, which exits from the axial beam scanning system 6.

In an exemplary embodiment, the first optical system 25 is controllably displaceable along the optical axis and the second optical system 26 is stationary. Thereby, for changing the scanning state of the axial scanning system 6, only the first optical system 25, which has a smaller effective diameter than the second optical system 26, is displaced for changing the scanning state of the axial scanning system. This even more increases the scanning velocity of the axial scanning system.

FIG. 5B is a schematic illustration of the beam deflection scanning system 7 of the eye treatment system according to the exemplary embodiment, which is configured to provide two angular scanning dimensions. The beam deflection scanning system 7 is a three mirror system. However, the present disclosure is not limited to such beam deflection scanning systems. Specifically, it is also conceivable that the beam deflection scanning system is a two mirror system in which each of the scanning mirrors is configured to provide one of the angular scanning dimensions.

In the three mirror scanning system, which is shown in FIG. 5B, two scanning mirrors 39 and 40 provide a first one of the angular scanning dimensions and a third scanning mirror 41 provides the second one of the angular scanning dimensions. Each of the scanning mirrors is rotatably mounted about a stationary rotation axis. FIG. 5B illustrates two scanning states of the beam deflection scanning system in which the laser beam is deflected by different amounts by the first and second scanning mirrors so that FIG. 5B shows two different beam paths 9a, 9b for the laser beam 9. The first and second scanning mirrors 39 and 40 are configured to deflect the scanning beam in a coordinated fashion so that the laser beam is pivoted about a pivoting point 42, which is located downstream of the first and second scanning mirrors 39, 40. Specifically, as is illustrated in FIG. 5B, the pivoting point 42 can be located on a surface of the third scanning mirror, in particular at a location, which at or substantially at the position of the rotation axis of the third scanning mirror 41.

It has been demonstrated by the inventors that such a three mirror configuration for the scanning system 7 reduces the field of curvature of the laser optical system and optimizes the spot size in the cornea. Thereby, it is possible to provide a contact surface of the contact element, which has a comparatively large radius of curvature so that during the surgical procedure, the intraocular pressure is kept within a range, which is acceptable for the patient.

FIG. 5C schematically illustrates a beam combiner 56 of the eye treatment system, which is also illustrated in FIG. 2. The beam combiner 56 is in the beam path of the laser beam 9 between the scanning system 43 and the contact element 10. The beam combiner 56 may be in the beam path of the laser beam between two components 8A and 8B of the focusing optical system, as it is illustrated in FIG. 5C. Each of the components 8A and 8B may include one or more optical elements, such as lenses. However, the present disclosure is not limited to such a configuration. It is also conceivable that the beam combiner 56 is either in the beam path of the laser beam 9 between the scanning system 43 and the focusing optical system 8 or in the beam path of the laser beam 9 between the focusing optical system 8 and the contact element 10.

The beam combiner may include a semi-transparent mirror or a prism. The semi-transparent mirror or prism may be configured as a dichroic mirror or prism. As it is schematically illustrated in FIGS. 2 and 5C, the beam combiner 56 is configured to combine the beam path of the laser beam 9 on the one hand with a measurement beam path 61 of an optical coherence tomography system 57 and an observation beam path 60 of a an imaging system 58 having a two-dimensional light sensitive imaging sensor on the other hand. The light sensitive image sensor may have a two-dimensional array of light sensitive pixels. The optical coherence imaging system may be configured to acquire cross-sectional images of the cornea and/or the natural lens of the eye. The imaging system, which has the imaging sensor may be configured to acquire a two-dimensional frontal image of the eye.

The controller of the eye treatment may be configured to determine, based on the acquired total image of the eye and/or based on a cross-sectional image acquired using the optical coherence system, a location and/or an orientation of the lamella, in particular a position and/or an orientation of the hinged flap relative to the eye.

In the eye treatment system according to the exemplary embodiment, the measurement beam path of the optical coherence system 57 and the observation beam path of the imaging system 58 are combined using a second beam combiner 59, which is outside the beam path of the pulsed laser beam 9. The second beam combiner may include a mirror or a prism.

Since the scanning system (designated with reference numeral 43 in FIG. 2) is in the beam path of the laser beam 9 upstream of the focusing optical system 8, it is possible to combine beam paths of the imaging system 58 and/or the optical coherence imaging system 57 with the beam path of the treatment laser beam 9, as it is illustrated in FIG. 5C.

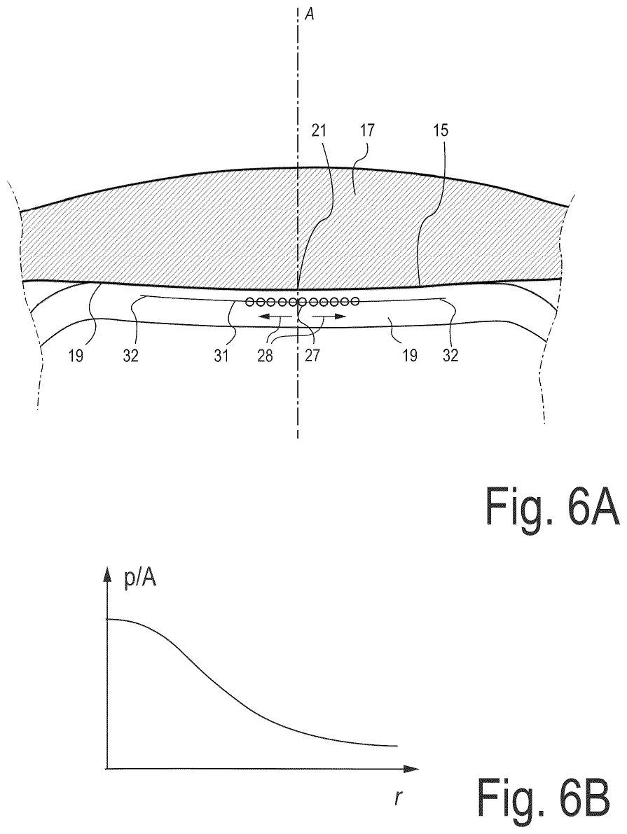

As will be explained in the following, the convex shape of the contact surface 15 has further advantageous effects when forming subsurface cuts within the cornea. As has been explained above, during formation of a subsurface cut within the cornea, gas bubbles which are caused by photodisruption of the corneal tissue can lead to undesirable effects such as opaque bubble layers, vertical gas breakthroughs or gas bubbles in the anterior chamber. However, the inventors have demonstrated that using the convex shape of the contact surface, it is possible to at least partially remove the gas bubbles from the subsurface cut and/or to move the gas bubbles toward a peripheral portion of the subsurface cut. This is explained in more detail with reference to FIGS. 6A to 12.

As is illustrated in FIGS. 6A and 6B, due to the convex shape of the contact surface 15, a local pressing force per unit area p/A, is generated on the anterior surface of the cornea, which decreases with increasing radial distance r from the apex 21 of the convex contact surface 15. The decrease of the local pressing force per unit area p/A with increasing radial distance r is schematically illustrated by the graph in FIG. 6B.

As a result of the decreasing pressing force per area p/A, the gas bubbles (schematically indicated in FIG. 6A with empty circles, such as for the gas bubble 27) are guided within the subsurface cut 31 in substantially radial directions 28 away from the apex 21 of the convex contact surface 15, which may be located on the optical axis A toward a peripheral portion 32 of the subsurface cut. Thereby, a central region of the subsurface cut 31, which is close to the apex 21, can be kept free from gas bubbles, which otherwise can lead to opaque bubble layers in the line of sight of the patient when generating the central region of the subsurface cut 31.

The inventors have further demonstrated that it is possible to remove the gas bubbles from the subsurface cut 31 using the contact element 10 having the convex contact surface 15.

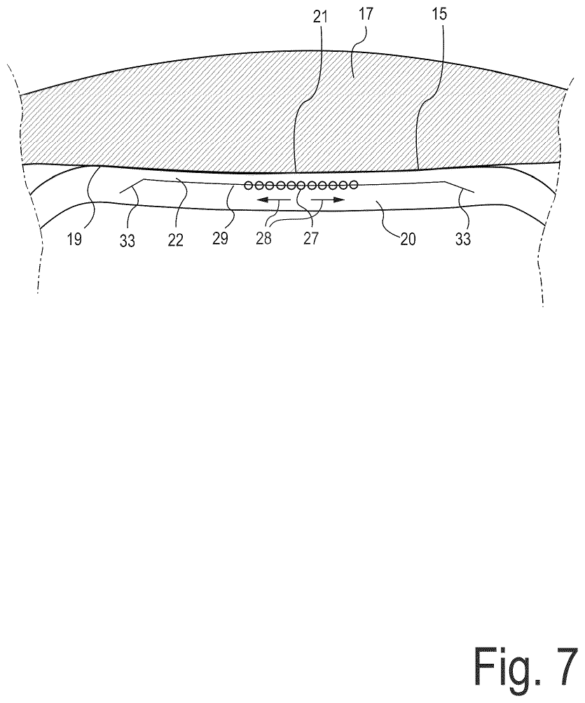

Specifically, as is illustrated in FIG. 7, the laser system may be configured to form a reservoir cut 33, which is a subsurface cut and which functions as reservoir for the gas bubbles, which are guided away from the central portion of the subsurface cut 31. The reservoir cut 33 may completely or partially surround the subsurface cut 31. The reservoir cut 33 may be in gas fluid communication with the subsurface cut 31. It is also conceivable that a plurality of reservoir cuts 33 are provided, which are circumferentially distributed about the subsurface cut 31. As is illustrated in FIG. 7, the reservoir cut 33 starts form a peripheral portion of the subsurface cut 31 and extends in a radial direction to deeper depths within the cornea. However, the present disclosure is not limited to such a configuration of the reservoir cut 33. It is also conceivable that the reservoir cut 33 extends in the radial direction to smaller depths or is located at a same or substantially same depth as the subsurface cut 31.

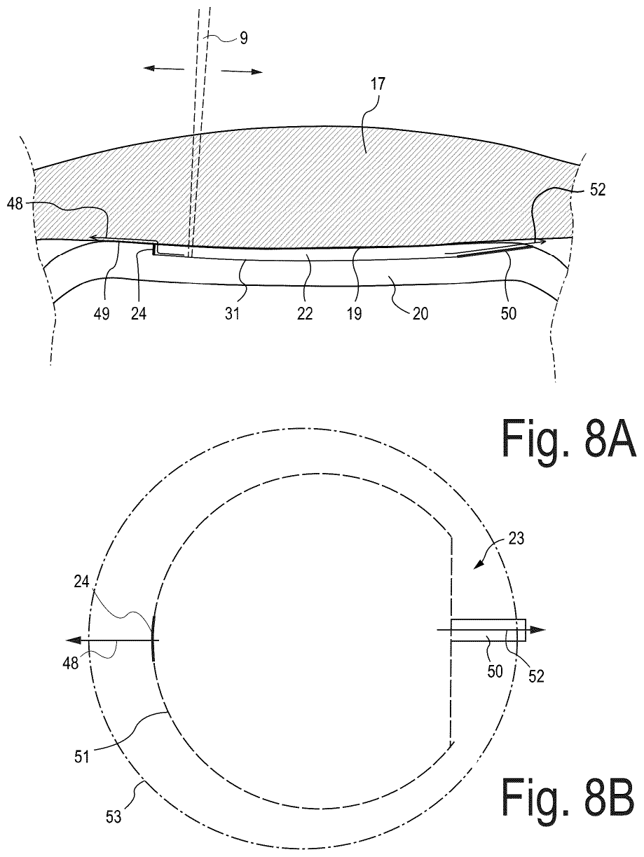

Additionally or alternatively, the eye treatment system is configured to generate gas release cuts, which are in fluid communication with the subsurface cut and which extend to the anterior surface of the cornea. Thereby, the gas can be released to the exterior of the eye. By way of example, as is illustrated in FIGS. 8A and 8B, in a process of forming the subsurface cut 31 for forming a flap bed of a hinged flap, before the subsurface cut 31 is formed, gas release cuts 24 and/or 50 are formed, which extend to the anterior surface 19 of the cornea 20.

FIG. 8A is a cross-sectional view taken along an optical axis of the eye and FIG. 8B is a top view, which illustrates features of the flap and the gas release cuts. The dashed line 51 represents the extent of the subsurface cut 31, which forms the stromal bed and the dash-dotted line 53 indicates the extent of the anterior surface 19 of the cornea 20, where the anterior surface 19 contacts the curved surface 15 of the contact element 10.

As can be seen from FIG. 8B, the gas release cut 50 is provided in the tissue region 23, which functions as the hinge of the flap. Since the gas release cut 50 opens into the anterior surface at a location, where the contact surface 15 does not contact the anterior surface 19 of the cornea 20, the gas bubbles are directly released to the surrounding atmosphere, as it is schematically indicated by arrow 52 in FIGS. 8A and 8B. The space between the distal surface of the lens-shaped element and the anterior surface 19 of the cornea, where the gas release cut 50 opens into the anterior surface 19 of the cornea, may be filled with a liquid (e.g. a physiological liquid, such as saline solution). The liquid allows obtaining a smaller focus diameter of the laser beam 9 when generating the gas release cut 50. However, sufficiently satisfactory results can be obtained without the liquid, i.e. when air is in the space between the distal surface of the lens-shaped element and the anterior surface 19 of the cornea, where the gas release cut 50 opens into the anterior surface 19. Alternatively, the gas release cut 50 may be configured so that the gas release cut 50 is in gas fluid communication with the anterior surface 19 of the cornea at a location where the contact surface 15 contacts the cornea 20, such as is illustrated in FIG. 8A for the gas release cut 24.

On the other hand, the gas release cut 24 is provided in a tissue region, which will later form the side cut of the flap and therefore opens into the anterior surface 19 of the cornea 20 at a location, where the contact surface 15 of the contact element 10 is in contact with the anterior surface 19 of the cornea 20.

For the gas release cut 24, it has been demonstrated by the inventors that due to the curved shape of the contact surface 15, it is possible to more efficiently release the gas to the surrounding atmosphere via the interface 49 between the anterior surface 19 and the contact surface 15, as it is illustrated by arrow 48 in FIGS. 8A and 8B. It is to be noted that for releasing the gas via the interface 49 to the exterior, it is not necessary for the contact surface to have a convex shape. However, the inventors have demonstrated that a convex surface of the contact surface allows more efficient release of the gases to the exterior.

It is to be noted that the shape of the gas release cuts 24, 50, which are shown in FIGS. 8A and 8B are only exemplary. Specifically, it is possible that the gas release cut 24 is not arranged perpendicularly relative to the anterior surface 19 of the cornea 20, but obliquely.

In a further exemplary embodiment, which is not illustrated in the Figures, the lamella is configured so that at least a portion of the posterior surface of the lamella is a portion of the posterior surface of the cornea. By way of example, such a lamella may be formed for performing laser endothelial keratoplasty procedures. The lamella may be a flap, which is partially isolated from the surrounding corneal tissue using the laser beam or a lenticule, which is completely isolated from the surrounding corneal tissue using the laser beam.

The lamella may be formed using a subsurface cut, which represents at least a portion of the anterior surface of the lamella and a side cut, which represents at least a portion of a rim of the lamella.

The controller of the eye treatment system may be configured to control the laser optical system to form one or more gas conducting gas release cuts in the cornea. Each of the gas release cuts extends to the anterior or to the posterior surface of the cornea. By way of example, a gas release cut, which extends to the anterior surface of the cornea may be configured as an access cut for access of surgical instruments, such as an instrument for separating the lenticule from the cornea and/or for removing the lenticule from the eye.

After formation of the one or more gas release cuts, at least a portion of the anterior surface of the lamella is formed using a subsurface cut. The gas release cuts, which extend to the anterior surface provide a gas conducting connection between the subsurface cut and the anterior surface of the cornea and the gas release cuts, which extend to the posterior surface provide a gas conducting connection between the subsurface cut and the posterior surface of the cornea.

Then, the side cut, which forms at least a portion of the rim of the lamella is completed after formation of the subsurface cut so that for each of the gas release cuts, at least a portion of the respective gas release cut forms a portion of the side cut.

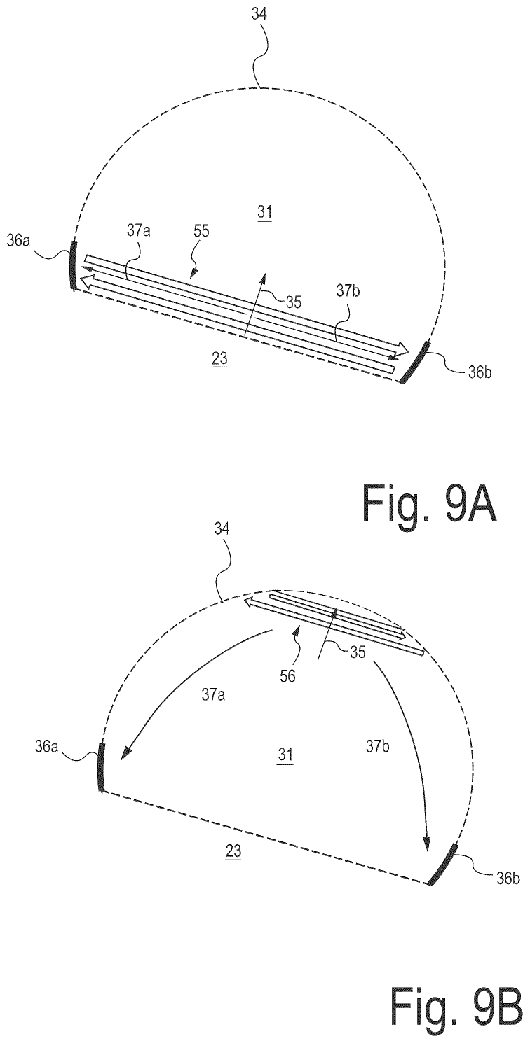

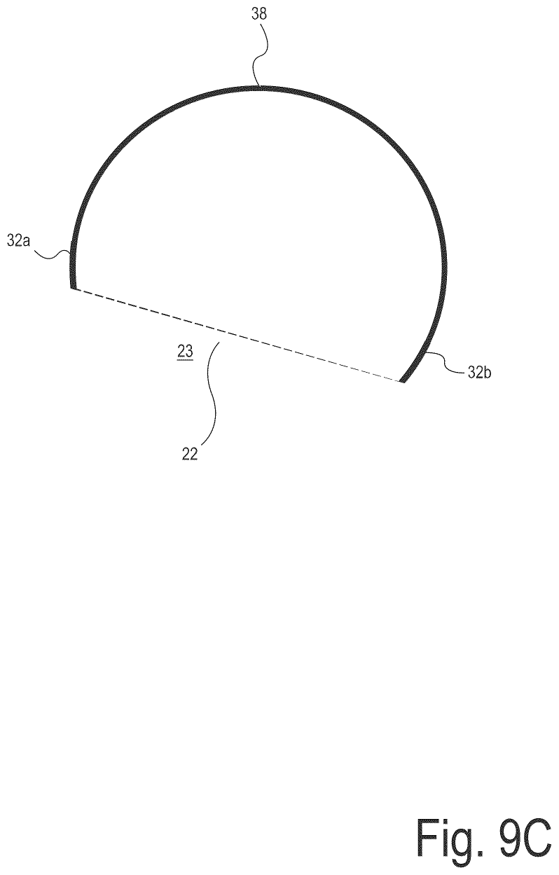

The formation of the one or more gas release cuts, which are arranged in a manner as the gas release cut 24 in FIGS. 8A and 8B is discussed in more detail in the following with reference to FIGS. 9A to 12. It is noted that although the description of FIGS. 9A to 12 relate to flaps, which represent an anterior surface lamella (such as LASIK flaps), the features of the description also apply to intrastromal lamella (such as in SMILE and FLEx procedures) and posterior surface lamella (such as in endothelial keratoplasty procedures). Accordingly, the effects advantages which are described in in connection with FIGS. 9A to 12 can also be obtained for gas release cuts, which extend to the posterior surface of the cornea so that gas is released from the subsurface cut into the anterior chamber of the eye. Each of FIGS. 9A to 9B represents a top view of the cornea in which the lateral extent of the subsurface cut 31, which forms the stromal bed is indicated by dashed line 34. The tissue portion where the hinge is located is indicated with reference numeral 23. The double line arrows represent scanning movements along a line scan direction of a meandering scanning pattern for forming the subsurface cut 31 using the beam deflection scanning system. The scanning direction of the scanning pattern for moving the laser focus between consecutive line scans is schematically indicated by arrow 35.

FIG. 9A schematically illustrates an initial section 55 of the raster scanning process and FIG. 9B schematically illustrates a final section of the raster scanning process. After formation of the subsurface cut 31, a side cut (designated with reference numeral 38 in FIG. 9C) is formed, which is connected with the subsurface cut 31 and which may extend to the anterior surface of the cornea over its full circumference. In alternative embodiments, the side cut extends into the epithelium without fully extending to the anterior surface of the cornea so that the portion of the side cut only substantially extends to the anterior surface of the cornea. No side cut is formed at the tissue region 23, which forms the hinge.

Returning to FIG. 9A, before the meandering or raster scanning process for forming the subsurface cut 31 is started, the laser system generates gas release cuts 36a and 36b. The gas release cuts 36a and 36b extend to the anterior surface of the cornea and are configured so that after formation of the subsurface cut 31, the subsurface cut 31 is in gas fluid communication with each of the gas release cuts 36a, 36b so that the gas release cuts 36a, 36b can release gases to the anterior surface of the cornea, which are generated through formation of the subsurface cut 31.

As is illustrated in FIG. 9A, the gas release cuts 36a and 36b are arranged so that substantially during the entire scanning process for forming the subsurface cut 31, gases, which are generated during formation of the subsurface cut 31, can be released to the exterior. This is schematically illustrated by arrows 37a and 37b in FIGS. 9A and 9B. Specifically, the controller of the eye treatment system is configured to control the laser optical system to form at least one of the gas release cuts 37a, 37b so that a location at which the gas release cut contacts the subsurface cut, the location is scanned by an initial section 55 of the scanning path, which corresponds to less than 60% or less than 50% or less than 30% or less than 20% of the total scanning path for forming the subsurface cut 31.

After formation of the subsurface cut 31, the eye treatment system forms the side cut 38, as is illustrated in FIG. 9C. As can be seen from FIG. 9C, the laser system is configured to form the side cut 38 so that the gas release cuts 32a and 32b form part of the side cut 38 of the flap. As can be seen by comparing FIGS. 9A and 9B on the one hand with FIG. 9C on the other hand, each of the gas release cuts 32a and 32b forms or comprises a circumferential portion of the side cut 38.

Therefore, after the flap has been formed in the cornea, no additional cuts remain, which were formed for releasing gas to the exterior during formation of the subsurface cut 31. It has been demonstrated by the inventors that this results in a higher stability for the cornea after formation of the flap. Specifically, since the gas release cuts form part of the later to be formed side cut 38, it is possible to form more gas release cuts or to form gas release cuts, which have a larger diameter so that the gases can more efficiently be released to the exterior without destabilizing the cornea. Thereby, the gas bubbles can be much more efficiently removed from the subsurface cut. As can be seen from FIGS. 9A and 9B, a combined circumferential length of the gas release cuts 36a and 36b (i.e. the sum of their circumferential length, as measured along a circumference of the side cut) is less than 60% or less than 50% of the circumferential length of the side cut 38.

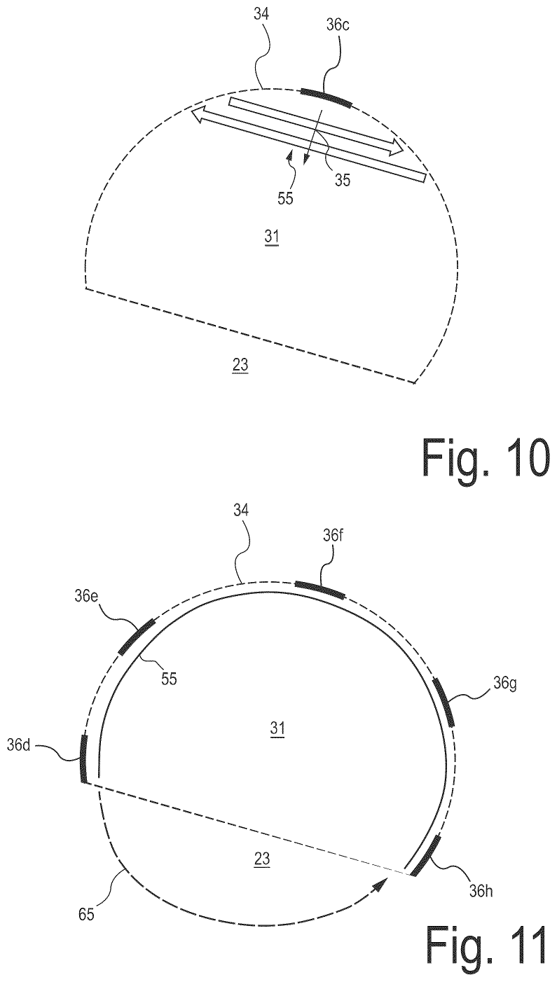

FIG. 10 shows a further exemplary embodiment of a flap formation process in which the initial section 55 of the raster scanning pattern is located at a lateral position, which is opposite to the hinge 23. The gas release cut 36c is therefore formed so that the gas release cut 36c contacts the subsurface cut 31 at at least one location, which is scanned by the initial section 55 of the scanning pattern, which corresponds to less than 60%, or less than 50%, or less than 30%, or less than 20% of the total scanning path for forming the subsurface cut 31.

FIG. 11 is a further exemplary embodiment of a flap formation process in which the subsurface cut is formed using a spiral scanning pattern. The initial section 55 of the scanning pattern (as defined above as a percentage of the total scanning path) forms a peripheral portion of the subsurface cut 31 and the scanning pattern progresses toward a center of the subsurface cut 31. Since the initial section of the scanning pattern forms the peripheral portion of the subsurface cut 31, each of the gas release cuts, 36d, 36e, 36f, 36g and 36h are located at the initial section 55 of the scanning path. The spiral scanning pattern further has the advantage that due to the curved contact surface 15 of the contact element 10, which has the apex (designated with reference numeral 21 in FIG. 3B), the gas bubbles are efficiently guided along radial paths to the peripheral portion of the subsurface cut 31. As can further be seen from FIG. 11, for example five gas release cuts are provided, which are connected in series by the later to be formed side cut. At a circumferential portion 65 of the spiral or ring scanning pattern, where the hinge 23 is located, the laser beam is blanked. It is however also conceivable that the release incisions are generated individually in a non-continuous scanning pattern.

The controller may be configured for automatically or interactively selecting of one of a plurality of pre-defined or configurable scanning patterns (such as one of the scanning patterns described in connection with FIGS. 9A to 11) for forming the subsurface cut 31. The controller may further be configured to determine, based on the selected scanning pattern, which is used for forming the subsurface cut: i) for at least one of the gas release cuts, a parameter of a) a geometrical shape, b) a position and/or c) an orientation. Additionally or alternatively, the controller may determine a number of the one or more gas release cuts.

By way of example, if the controller, interactively or automatically, selects the scanning pattern of FIG. 10, the controller determines that a single gas release cut, such as the gas release cut 36c, which is shown in FIG. 10 is provided, which is located at the initial section of the scanning pattern. Further by way of example, if the controller, automatically or interactively selects the scanning pattern of FIGS. 9A and 9B, the controller determines that two gas release cuts are provided (such as the gas release cuts 36a and 36b, which are shown in FIGS. 9A and 9B), each of which being provided at or proximate to the hinge 23.

The scanning patterns may be represented by one or more data structures based on which the eye treatment system generates control signals for controlling the scanning system. The data structures may be configurable using user input, which is received via a user interface of the eye treatment system. The user input may include one or more parameters of the scanning pattern, such as the line-to-line spacing of a scanning pattern (e.g. the line-to-line spacing of a meandering scanning pattern) and/or the starting point of the scanning pattern.

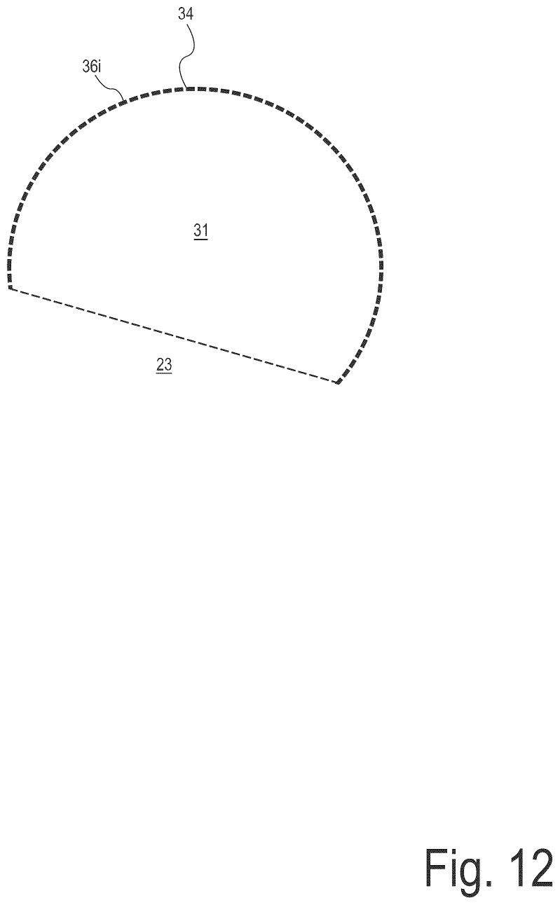

FIG. 12 is a still further exemplary embodiment of the flap formation process in which a large number of gas release cuts, such as the gas release cut 36i, are formed. After formation of the subsurface cut 31, the gas release cuts are circumferentially distributed about the periphery of the flap bed cut. Each of the gas release cuts has a peripheral extent, which is less than 40 degrees or less than 20 degrees or less than 10 degrees. Further, the number of the gas release cuts is comparatively large. By way of example, the number of the gas release cuts is larger than 5 or larger than 10 or larger than 20 or larger than 50. The inventors have demonstrated that the comparatively large number of gas release cuts, facilitates release of the gases to the exterior. Specifically, since there is a large number of gas release cuts, which are circumferentially distributed about the outer periphery of the subsurface cut 31, gas bubbles, which migrate in a radial direction have a comparatively high probability of being directed directly to one of the gas release cuts. Further, the combined cross-section of the gas release cuts is comparatively large. After formation of the subsurface cut 31, the gas release cuts are connected to form the side cut, as is illustrated in FIG. 9C.

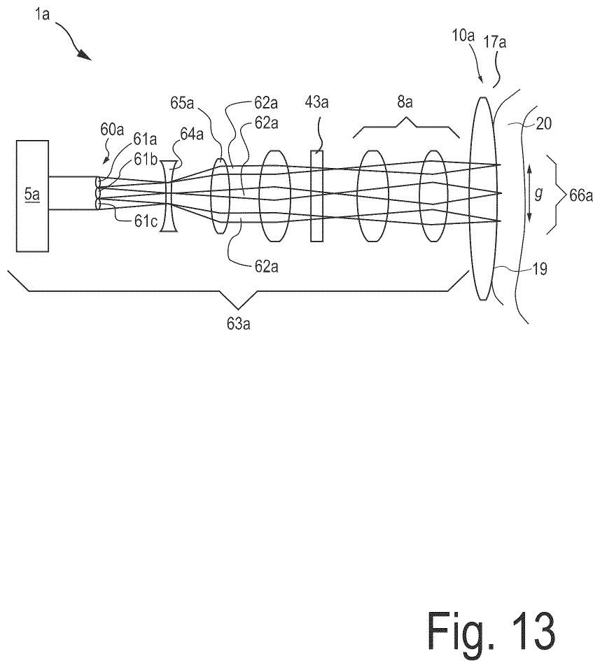

FIG. 13 is a schematic illustration of an eye treatment system 1a according to a second exemplary embodiment. Elements in the second exemplary embodiment of FIG. 13, which are similar to those of the first exemplary embodiment, which are illustrated in FIGS. 1 to 5C, have been identified by like reference numerals with the suffix letter “a” being employed to distinguish the elements of the second exemplary embodiment from the corresponding elements of the first exemplary embodiment.

The eye treatment system 1a of the second exemplary embodiment has a laser optical system 63a, which includes a beam multiplier 60a, which is arranged downstream of the laser source 5a to receive pulsed laser light emitted by the laser source 5a. The beam multiplier 60a is configured to generate a plurality of beams 62a. As can be seen from FIG. 13, the laser optical system 63a is configured to generate three beams. However, the present disclosure is not limited to eye treatment systems, which generate three beams. Specifically, it is conceivable that the eye treatment system 1a is configured to generate 2, 4 or more beams. The number of beams 62a may be a less than 500, or less than 200, or less than 100. When seen in a plane, which is perpendicular to the optical axis of the laser surgical system 63a, the beams may represent an ordered or disordered one-dimensional, two-dimensional or three-dimensional array.

The beam multiplier 60a is configured as an ordered or disordered array of lenses, such as an array of microlenses. The principal planes of the lenses are arranged or substantially arranged in a common plane. Therefore, the lenses are illuminated in parallel (i.e. not successively) by the incoming laser beam 9a. It is to be noted that the second exemplary embodiment is not limited to such a configuration of the beam multiplier 60a. Specifically, additionally or alternatively, the beam multiplier 60a may include an ordered or disordered array of mirrors, in particular an array of micro-mirrors. The mirrors may be arranged in the beam path of the pulsed laser so that they are illuminated in parallel by the incoming pulsed laser beam. Additionally or alternatively, the beam multiplier 60a may include a phase mask or a spatial light modulator (SLM).

As is further illustrated in FIG. 13, the laser optical system 63a includes a scanning system 43a for scanning each of the beams 62a within the cornea 20 of the patient's eye in three dimensions. The scanning system 43a may include an axial scanning system and a beam deflection scanning system (which are not illustrated in FIG. 13). Similarly as has been described in connection with the first exemplary embodiment, the beam deflection scanning system may include two or three mirrors, wherein each of the scanning mirrors receives each of the beams 62a so that the beams are synchronously angularly deflected in two angular dimensions by the beam deflection scanning system. The axial scanning system may include, for each of the beams 62a, a first optical system and a second optical system, which are displaceable relative to each other so that a distance between the first and second optical system is controllably variable. The first and second optical system may be configured in a manner as has been described in connection with the first exemplary embodiment. Specifically, the first optical system may have a negative optical power and the second optical system may have a positive optical power.

By way of example, the axial scanning system may include a first array of lenses, which provides, for each of the beams 62a, the first optical system. Further, the axial scanning system may include a second array of lenses, which provides for each of the beams 62a, the second optical system. The first and second arrays of lenses are controllably displaceable relative to each other so that for each of the beams 62a, the distance between the first and second optical system is controllably varied. The eye treatment system 1 when including the laser optical system 63a (from here on referred to as 1a) may also include an actuator for controllably varying a distance between the first and second arrays of lenses of the axial scanning system so that for each of the beams 62a, a focus of the respective beam is displaced along an axis of the respective beam. Thereby, it is possible to synchronously axially scan the foci of the beams 62a within the cornea 20. In the eye treatment system 1a according to the second exemplary embodiment, the axial scanning system may be in the pulsed laser light between the laser source 5a and the beam deflection scanning system. However, the disclosure is not limited to such a configuration of the scanning system 43a and it is also conceivable that the beam deflection scanning system is in the pulsed laser light between the laser source 5a and the axial scanning system.

Therefore, the scanning system 43a of the eye treatment system 1a is configured to synchronously scan the foci of the beams 62a within the cornea of the eye in three dimensions.

As can further be seen from FIG. 13, between the laser source 5a and the scanning system 43a, a negative field lens 64a is disposed in the beam path of the beams 62a. The negative field lens 64a is configured so that the sections of the beams 62a, which exit from the negative field lens 64a are mutually divergent. The mutually divergent sections of the beams 62a, which exit from the negative field lens 64a are incident on a collimating lens 65a, which is configured to convert the mutually divergent sections into mutually parallel sections of the beams 62a. However, it has been shown that an eye treatment system with sufficient performance can be obtained without the field lens 64a and the collimating lens 65a.

As can further be seen from FIG. 13, a contact element 10a is arranged in the beam path between the laser optical system 63a and the cornea 20 of the patient's eye. For simplicity of illustration, only the lens-shaped portion 17a of the contact element 10a is shown in FIG. 13. The configuration and of the contact element 10a of the eye treatment system of the second exemplary embodiment and the manner in which the contact element 10a is coupled relative to the laser optical system 63a and the cornea 20 of the eye corresponds to the configuration and variants, as has been explained in connection with the eye treatment system 1a of the first exemplary embodiment.

As has been explained in connection with the eye treatment system 1a of the first exemplary embodiment, the contact element 10a is configured so that, for each of the beams 62a, the contact element 10a reduces the variation of the depth of at least a portion of the scanning plane of the focus of the respective beam, wherein the scanning plane corresponds to a constant scanning state of the axial scanning system.

The eye treatment system 1a of the second exemplary embodiment is configured so that the scanning planes of the beams 62a are substantially identical or match the slope of the side cut or gas release or reservoir cuts to be created. In each of these configurations it is essential for a smooth cut surface and reasonable speed to effectively compensate the field curvature which otherwise would offset the points relative to each other in a manner depending on the current distance from the apex. Therefore, it has been shown that, using a contact element having a convex contact surface, for each of the beams and of the beams relative to each other, a depth variation of the scanning plane of the respective laser beam can efficiently be reduced.

Therefore, the eye treatment system 1a of the second exemplary embodiment allows even much faster formation of subsurface cuts within the cornea.

The eye treatment system may be configured to be switchable between a multifocal mode for forming the subsurface cut and a single focus mode for forming the side cut. Specifically, the eye treatment system may be configured so that the switching includes selectively inserting or retracting an aperture and/or the beam multiplier into or from the beam path.

Additionally or alternatively, the eye treatment system may be configured to form gas release cuts and/or side cuts using a plurality of foci. Specifically, the beam multiplier may be configured to generate a line of foci, wherein the foci are arranged or are substantially arranged on a straight or curved line which extends at a constant depth, wherein the depth can be adjusted using the axial scanning system. By way of example, the foci may be arranged substantially on a curved line so that a radius of curvature of the curved line corresponds to the radius of curvature of the side cut. By way of example, the radius of curvature of the curved line and the radius of curvature of the side cut have a value, which is within a range of between 4 and 4.9 millimeters.

The eye treatment system may include a rotatably mounted phase plate, a micromirror array and/or a microlens array for adjusting an orientation of the line of foci within the focal plane. When forming the side cut, the eye treatment system may be configured to adjust the orientation of the line of foci to different circumferential positions of the side cut so that a circular or substantially circular side cut is generated.

In an alternative embodiment, the foci are located at different depths so that the side cut can be formed simultaneously at different depths. Also in this alternative embodiment, the eye treatment system may include a phase plate, micromirror array and/or microlens array for adjusting an orientation of the regular or irregular array of foci relative to an axis, which extends along or parallel to the optical axis of the laser system so that the orientation of the array of foci is adjustable to different circumferential positions of the side cut.

Before we go on to set out the claims, we first set out the following clauses describing some prominent features of certain embodiments of the present disclosure:
1. An eye treatment system for performing laser surgery on an eye, comprising: a laser optical system having a laser source configured to generate pulsed laser light having a pulse duration of less than 1 picoseconds; wherein the laser optical system comprises: a scanning system for scanning a focus of a laser beam of the laser light within a cornea of the eye in three dimensions; and a focusing optical system, wherein the scanning system is in the beam path of the laser beam between the laser source and the focusing optical system; wherein the eye treatment system further comprises a contact element which is in the beam path of the laser beam between the focusing optical system and the eye; wherein the contact element has a contact surface for contacting a cornea of the eye wherein at least a portion of the contact surface has a shape, which is convex toward the cornea.
2. The eye treatment system of clause 1, wherein the scanning system comprises: an axial scanning system for scanning the laser focus along an axis of the laser beam; and a beam deflection scanning system for scanning the laser beam through deflection of the laser beam.
3. The eye treatment system of clause 1 or 2, wherein, compared to a plane-parallel applanation plate, the contact element is configured to reduce a variation of a depth of at least a portion of a scanning plane of the laser focus; wherein the depth is measured relative to the anterior surface and the scanning plane corresponds to a constant scanning state of an axial scanning system of the scanning system for scanning the laser focus along an axis of the laser beam.
4. The eye treatment system of any one of the preceding clauses 1 to 3, wherein the contact element is configured so that a depth variation of a scanning plane of the laser focus is less than 30 micrometers, or less than 20 micrometers, at least for each point having a distance from an optical axis of the laser optical system which is less than 2 millimeters or less than 4 millimeters, or less than 5.5 millimeters, or less than 6 millimeters; wherein the scanning plane corresponds to a constant scanning state of an axial scanning system of the eye treatment system, which is configured for scanning the laser focus along an axis of the laser beam.
5. The eye treatment system of any one of clauses 2 to 4, wherein the axial scanning system is in the beam path of the laser beam between the laser source and the beam deflection scanning system.
6. The eye treatment system of any one of the preceding clauses 2 to 5, wherein the axial scanning system includes: a first optical system, which has a negative optical power; and a second optical system, which has a positive optical power; wherein the second optical system is in the beam path of the laser beam between the first optical system and the deflection scanning system; wherein the axial scanning system is configured so that a distance between the first optical system and the second optical system is controllably variable.
7. The eye treatment system of any one of the preceding clauses 1 to 6, wherein eye treatment system includes a beam deflecting scanning system for scanning the laser beam through deflection of the laser beam, wherein the beam deflection scanning system comprises three scanning mirrors.
8. The eye treatment system of any one of the preceding clauses 1 to 7, wherein the laser system is configured so that the contact element is detachably coupleable relative to the laser optical system; wherein the eye treatment system is configured so that the contact element is the only optical element, which is in the beam path of the laser beam between the laser optical system and the eye.
9. The eye treatment system of any one of the preceding clauses 1 to 8, wherein the laser optical system comprises beam combiner, which is in the beam path of the laser beam between the scanning system and the eye.
10. The eye treatment system of any one of the preceding clauses 1 to 9, wherein the eye treatment system comprises a beam combiner which is configured for combining the beam path of the laser beam with a beam path of an imaging system of the eye treatment system.
11. The eye treatment system of any one of the preceding clauses 1 to 10, wherein at least a portion of the convex shape of the contact surface has a radius of, curvature, which is: greater than 10 millimeters, or greater than 50 millimeters; and/or smaller than 500 millimeters, or smaller than 300 millimeters.
12. The eye treatment system of any one of the preceding clauses 1 to 11, wherein the contact surface has a convex shape at least for each point having a distance from an apex of the contact surface of less than 2 millimeters, or less than 4 millimeters, or less than 6 millimeters.
13. The eye treatment system of any one of the preceding clauses 1 to 12, wherein the laser system is configured so that the contact element is detachably coupleable relative to the focusing optical system, wherein in the coupled state, the contact element is in a predefined radial position relative to an optical axis of the focusing optical system and/or a predefined inclination relative to the optical axis of the laser optical system.
14. The eye treatment system of any one of the preceding clauses 1 to 13, wherein the laser optical system is configured to generate, using the laser beam, a substantially laterally extending subsurface cut within the cornea; wherein the convex shape is configured so that gas, which is caused by the formation of the substantially laterally extending subsurface cut, is guided substantially in a direction away from an apex of the convex shape.
15. The eye treatment system of any one of the preceding clauses 1 to 14, wherein the laser beam is one of a plurality of laser beams, which are generated by the laser optical system using a beam multiplier of the eye treatment system, wherein the beam multiplier is arranged so that the pulsed laser light impinges on the beam multiplier and the scanning system is configured to scan an ordered or disordered one-, two or three-dimensional focus array within the cornea, which is generated by the laser optical system using the plurality of laser beams.
16. The eye treatment system of any one of the preceding clauses 1 to 15, wherein the laser system is configured for forming a lamella of corneal tissue, in particular a corneal flap an intracorneal lenticule, or a corneal surface lamella; wherein the eye treatment system comprises a controller, which is configured to control the laser optical system to scan the focus within the cornea to at least partially isolate the lamella from surrounding corneal tissue using a subsurface cut and a side cut; wherein the subsurface cut represents at least a portion of an anterior or posterior surface of the lamella and the side cut represents at least a portion of a rim of the lamella, wherein for forming the lamella, the controller is configured to control the laser optical system to: form one or more gas conducting gas release cuts in the cornea, wherein for each of the gas release cuts, the respective cut extends to an anterior surface or posterior surface of the cornea and at least a portion of the gas release cut forms at least a portion of the rim of the lamella; to form at least a portion of the subsurface cut after formation of the one or more gas release cuts; and to complete the side cut after formation of the subsurface cut so that for each of the gas release cuts, at least a portion of the respective gas release cut forms a portion of the side cut.
17. An eye treatment system for forming a lamella of corneal tissue, in particular a corneal flap, an intracorneal lamella, or a corneal surface lamella, the eye treatment system comprising: a laser optical system having a laser source configured to generate a pulsed laser beam having a pulse duration of less than 1 picoseconds; and a controller which is configured to control the laser optical system to scan a focus of the laser beam within a cornea to at least partially isolate the lamella from surrounding corneal tissue using a subsurface cut and a side cut; wherein the subsurface cut represents at least a portion of an anterior or posterior surface of the lamella and the side cut represents at least a portion of a rim of the lamella; wherein for forming the lamella, the controller is configured to control the laser optical system to: form one or more gas conducting gas release cuts in the cornea, wherein for each of the gas release cuts, the respective cut extends to an anterior or posterior surface of the cornea and at least a portion of the gas release cut forms at least a portion of the rim of the lamella; to form at least a portion of the subsurface cut after formation of the one or more gas release cuts; and to complete the side cut after formation of the subsurface cut so that for each of the gas release cuts, at least a portion of the respective gas release cut forms a portion of the side cut.
18. The eye treatment system of clause 16 or 17, wherein for one or more or each of the gas release cuts, the respective gas release cut comprises a circumferential portion of the side cut.
19. The eye treatment system of any one of clauses 16 to 18, wherein at least one of the one or more of the gas release cuts has a circumferential extent, which is: less than 120 degrees or less than 90 degrees; and/or greater than 5 degrees or greater than 10 degrees.
20. The eye treatment system of any one of clauses 16 to 19, wherein after completion of the side cut, for each gas release cut, which was used for releasing gas from the subsurface cut, at least a portion of the respective gas release cut is a part of the side cut.
21. The eye treatment system of any one of clauses 16 to 20, wherein the lamella is a hinged flap and the side cut is the rim of the hinged flap.
22. The eye treatment system of any one of clauses 16 to 21, wherein the side cut is connected to the subsurface cut at a peripheral portion of the subsurface cut.
23. The eye treatment system of any one of clauses 16 to 22, wherein a combined circumferential length of the one or more gas release cuts, as measured along a circumference of the side cut, amounts to less than 60% or less than 50% of a circumferential length of the side cut.
24. The eye treatment system of any one of clauses 16 to 23, wherein forming the subsurface cut comprises automatically or interactively selecting one of a plurality of pre-defined scanning patterns for forming the subsurface cut; wherein the controller is further configured to determine, based on the selected scanning pattern, which is used for forming the subsurface cut: i) for at least one of the gas release cuts, a parameter of a) a geometrical shape, b) a position and/or c) an orientation and/or ii) a number of the one or more gas release cuts.
25. The eye treatment system of any one of clauses 16 to 24, wherein the controller is configured to control the laser optical system to form at least one of the gas release cuts so that the gas release cut contacts the subsurface cut at at least one location, which is scanned by an initial section of the scanning path, which corresponds to less than 60% or less than 50% of the total scanning path for forming the subsurface cut.
26. The eye treatment system of any one of clauses 16 to 25, wherein, measured along a circumference of the side cut, each of the gas release cuts has a circumferential extent which is less than 40 degrees or less than 20 degrees or less than 10 degrees; and wherein a number of the gas release cuts is larger than 5 or larger than 10 or larger than 20 or larger than 50.
27. The eye treatment system of any one of clauses 16 to 26, wherein the eye treatment system comprises a contact element, which is in the beam path of the laser beam between the laser optical system and the eye, the contact element having a contact surface, which contacts the cornea and which is convex toward the cornea to form an apex; wherein each of the gas release cuts opens into an anterior surface of the cornea at a location where the contact surface contacts the cornea.
28. An eye treatment system for forming a lamella of corneal tissue, in particular a corneal flap an intracorneal lamella, or a corneal surface lamella; wherein the eye treatment system comprises: a laser optical system having a laser source configured to generate a pulsed laser beam having a pulse duration of less than 1 picoseconds; a contact element, which is in the beam path of the laser beam between the laser optical system and the eye, wherein the contact element has a contact surface, which contacts the cornea, wherein the contact surface is convex toward the cornea to form an apex; a controller, which is configured to control the laser optical system to scan a focus of a laser beam within a cornea when the contact element is contact with the cornea to at least partially isolate the lamella from surrounding corneal tissue using a subsurface cut; wherein the controller is further configured to control the laser optical system to form one or more gas release cuts in the cornea before forming the subsurface cut, wherein each of the gas release cuts is in gas fluid communication with an anterior surface of the cornea at a location where the contact surface contacts the cornea.
29. The eye treatment system according to clause 27 or 28, wherein the laser optical system comprises a scanning system for scanning a focus of the laser beam within the eye in three dimensions; and wherein the scanning system comprises: an axial scanning system for scanning the laser focus along an axis of the laser beam; and a beam deflection scanning system for scanning the laser beam through deflection of the laser beam.
30. The eye treatment system according to any one of clauses 27 to 29, wherein compared to a plane-parallel applanation plate, the contact element is configured to reduce a variation of a depth of at least a portion of a scanning plane of the laser focus; wherein the depth is measured relative to the anterior surface and the scanning plane corresponds to a constant scanning state of an axial scanning system of the eye treatment system which is configured for scanning the laser focus along an axis of the laser beam.
31. The eye treatment system of any one of clauses 27 to 30, wherein the contact element is configured so that the depth variation of at least a portion of a scanning plane of the laser focus is less than 30 micrometers, or less than 20 micrometers, at least for each point having a distance from an optical axis of the laser optical system which is less than 2 millimeters or less than 4 millimeters, or less than 5.5 millimeters, or less than 6 millimeters, wherein the scanning plane corresponds to a constant scanning state of an axial scanning system of the eye treatment system for scanning the laser focus along an axis of the laser beam.
32. The eye treatment system of any one of causes 27 to 31, wherein the laser optical system comprises a scanning system for scanning the focus of the laser beam within the cornea, wherein the scanning system is in the beam path of the laser beam between the focusing optical system and the eye.
33. The eye treatment system of any one of clauses 27 to 32, wherein at least a portion of the convex shape has a radius of curvature, which is: greater than 10 millimeters or greater than 50 millimeters; and/or smaller than 500 millimeters or smaller than 300 millimeters.
34. The eye treatment system of any one of clauses 27 to 33, wherein the convex shape is configured so that gas, which is generated during formation of the subsurface cut is guided substantially in a direction away from an apex of the convex shape.
35. The eye treatment system of any one of clauses 1 to 34, wherein the contact element includes a proximal surface, which is in the beam path of the laser beam and which is opposite to the contact surface, wherein least a portion of the proximal surface has a shape, which is convex or concave toward the incident laser beam or which is planar.
36. A method of treating an eye for forming a lamella of corneal tissue, in particular a corneal flap an intracorneal lamella, or a corneal surface lamella, using: a laser optical system having a laser source configured to generate a pulsed laser beam having a pulse duration of less than 1 picoseconds and a scanning system for scanning a focus of the laser beam within a cornea to at least partially isolate the lamella from surrounding corneal tissue using a subsurface cut and a side cut; wherein the subsurface cut represents at least a portion of an anterior or posterior surface of the lamella and the side cut represents at least a portion of the rim of the lamella; wherein the method comprises: forming, using the laser optical system, one or more gas conducting gas release cuts in the cornea, wherein for each of the gas release cuts, the respective cut extends to an anterior or posterior surface of the cornea; forming, using the laser optical system, at least a portion of the subsurface cut after formation of the one or more gas release cuts; and completing, using the laser optical system, the side cut after formation of the subsurface cut so that for each of the gas release cuts, at least a portion of the respective gas release cut forms a portion of the side cut.

The above embodiments as described are only illustrative, and not intended to limit the technique approaches of the present invention. Although the present invention is described in details referring to the preferable embodiments, those skilled in the art will understand that the technique approaches of the present invention can be modified or equally displaced without departing from the protective scope of the claims of the present invention. In the claims, the word “comprising” does not exclude other elements or steps, and the indefinite article “a” or “an” does not exclude a plurality. Any reference signs in the claims should not be construed as limiting the scope.

1. An eye treatment system for performing laser surgery on an eye, comprising:
a laser optical system having a laser source configured to generate pulsed laser light having a pulse duration of less than 1 picoseconds;
wherein the laser optical system comprises:
a scanning system for scanning a focus of a laser beam of the laser light within a cornea of the eye in three dimensions; and
a focusing optical system, wherein the scanning system is in the beam path of the laser beam between the laser source and the focusing optical system;
wherein the eye treatment system further comprises a contact element which is in the beam path of the laser beam between the focusing optical system and the eye;
wherein the contact element has a contact surface for contacting a cornea of the eye wherein at least a portion of the contact surface has a shape, which is convex toward the cornea.

2. The eye treatment system of claim 1, wherein the scanning system comprises:
an axial scanning system for scanning the laser focus along an axis of the laser beam; and
a beam deflection scanning system for scanning the laser beam through deflection of the laser beam.

3. The eye treatment system of claim 2, wherein, compared to a plane-parallel applanation plate, the contact element is configured to reduce a variation of a depth of at least a portion of a scanning plane of the laser focus;
wherein the depth is measured relative to the anterior surface and the scanning plane corresponds to a constant scanning state of the axial scanning system.

4. The eye treatment system of claim 3, wherein the contact element is configured so that the depth variation is less than 30 micrometers, or less than 20 micrometers, at least for each point having a distance from an optical axis of the laser optical system which is less than 2 millimeters or less than 4 millimeters, or less than 5.5 millimeters, or less than 6 millimeters.

5. The eye treatment system of claim 2, wherein the axial scanning system is in the beam path of the laser beam between the laser source and the beam deflection scanning system.

6. The eye treatment system of claim 2, wherein the axial scanning system comprises:
a first optical system, which has a negative optical power; and
a second optical system, which has a positive optical power;
wherein the second optical system is in the beam path of the laser beam between the first optical system and the deflection scanning system;
wherein the axial scanning system is configured so that a distance between the first optical system and the second optical system is controllably variable.

7. The eye treatment system (1) of claim 1, wherein the eye treatment system includes a beam deflecting scanning system for scanning the laser beam through deflection of the laser beam, wherein the beam deflection scanning system comprises three scanning mirrors.

8. (canceled)

9. The eye treatment system of claim 1, wherein the laser optical system comprises a beam combiner, which is in the beam path of the laser beam between the scanning system and the eye, wherein the beam combiner is configured for combining the beam path of the laser beam with a beam path of an imaging system of the eye treatment system.

10. (canceled)

11. The eye treatment system of claim 1, wherein at least a portion of the convex shape of the contact surface has a radius of, curvature, which is:
greater than 10 millimeters, or greater than 50 millimeters; and/or
smaller than 500 millimeters, or smaller than 300 millimeters.

12. The eye treatment system of claim 1, wherein the contact surface has a convex shape at least for each point having a distance from an apex of the contact surface of less than 2 millimeters, or less than 4 millimeters, or less than 6 millimeters.

13-16. (canceled)

17. An eye treatment system for forming a lamella of corneal tissue, in particular a corneal flap, an intracorneal lamella, or a corneal surface lamella, the eye treatment system comprising:
a laser optical system having a laser source configured to generate a pulsed laser beam having a pulse duration of less than 1 picoseconds; and
a controller which is configured to control the laser optical system to scan a focus of the laser beam within a cornea to at least partially isolate the lamella from surrounding corneal tissue using a subsurface cut and a side cut;
wherein the subsurface cut represents at least a portion of an anterior or posterior surface of the lamella and the side cut represents at least a portion of a rim of the lamella;
wherein for forming the lamella, the controller is configured to control the laser optical system to:
form one or more gas conducting gas release cuts in the cornea, wherein for each of the gas release cuts, the respective cut extends to an anterior or posterior surface of the cornea and at least a portion of the gas release cut forms at least a portion of the rim of the lamella; to
form at least a portion of the subsurface cut after formation of the one or more gas release cuts; and to
complete the side cut after formation of the subsurface cut so that for each of the gas release cuts, at least a portion of the respective gas release cut forms a portion of the side cut.

18. The eye treatment system of claim 17, wherein for one or more or each of the gas release cuts, the respective gas release cut comprises a circumferential portion of the side cut.

19. The eye treatment system of claim 17, wherein at least one of the one or more of the gas release cuts has a circumferential extent, which is: less than 120 degrees or less than 90 degrees; and/or greater than 5 degrees or greater than 10 degrees.

20. The eye treatment system of any one of claim 17, wherein after completion of the side cut, for each gas release cut, which was used for releasing gas from the subsurface cut, at least a portion of the respective gas release cut is a part of the side cut.

21. The eye treatment system of any one of claim 17, wherein the lamella is a hinged flap and the side cut is the rim of the hinged flap.

22-27. (canceled)

28. An eye treatment system for forming a lamella of corneal tissue, in particular a corneal flap an intracorneal lamella, or a corneal surface lamella;
wherein the eye treatment system comprises:
a laser optical system having a laser source configured to generate a pulsed laser beam having a pulse duration of less than 1 picoseconds; a contact element, which is in the beam path of the laser beam between the laser optical system and the eye, wherein the contact element has a contact surface, which contacts the cornea, wherein the contact surface is convex toward the cornea to form an apex;
a controller, which is configured to control the laser optical system to scan a focus of a laser beam within a cornea when the contact element is contact with the cornea to at least partially isolate the lamella from surrounding corneal tissue using a subsurface cut;
wherein the controller is further configured to control the laser optical system to form one or more gas release cuts in the cornea before forming the subsurface cut,
wherein each of the gas release cuts is in gas fluid communication with an anterior surface of the cornea at a location where the contact surface contacts the cornea.

29. (canceled)

30. The eye treatment system according to claim 28, wherein compared to a plane-parallel applanation plate, the contact element is configured to reduce a variation of a depth of at least a portion of a scanning plane of the laser focus; wherein the depth is measured relative to the anterior surface and the scanning plane corresponds to a constant scanning state of an axial scanning system of the eye treatment system which is configured for scanning the laser focus along an axis of the laser beam.

31. The eye treatment system of claim 28, wherein the contact element is configured so that the depth variation of at least a portion of a scanning plane of the laser focus is less than 30 micrometers, or less than 20 micrometers, at least for each point having a distance from an optical axis of the laser optical system which is less than 2 millimeters or less than 4 millimeters, or less than 5.5 millimeters, or less than 6 millimeters, wherein the scanning plane corresponds to a constant scanning state of an axial scanning system of the eye treatment system for scanning the laser focus along an axis of the laser beam.

32-33. (canceled)

34. The eye treatment system of any one of claim 28, wherein the convex shape is configured so that gas, which is generated during formation of the subsurface cut is guided substantially in a direction away from an apex of the convex shape.

35. (canceled)

36. A method of treating an eye for forming a lamella of corneal tissue, in particular a corneal flap an intracorneal lamella, or a corneal surface lamella, using:
a laser optical system having a laser source configured to generate a pulsed laser beam having a pulse duration of less than 1 picoseconds and a scanning system for scanning a focus of the laser beam within a cornea to at least partially isolate the lamella from surrounding corneal tissue using a subsurface cut and a side cut;
wherein the subsurface cut represents at least a portion of an anterior or posterior surface of the lamella and the side cut represents at least a portion of the rim of the lamella;
wherein the method comprises:
forming, using the laser optical system, one or more gas conducting gas release cuts in the cornea, wherein for each of the gas release cuts, the respective cut extends to an anterior or posterior surface of the cornea;
forming, using the laser optical system, at least a portion of the subsurface cut after formation of the one or more gas release cuts; and
completing, using the laser optical system, the side cut after formation of the subsurface cut so that for each of the gas release cuts, at least a portion of the respective gas release cut forms a portion of the side cut.
